(19) United States
(12) Patent Application Publication
(10) Pub. No.: US 2011/0319937 A1
(43) Pub. Date: Dec. 29, 2011

(54) Side-loading interspinous process spacer an installation tool

(76) Inventor: Roger P. Jackson, Prairie Village, KS (US)

(21) Appl. No.: 13/199,461

(22) Filed: Aug. 31, 2011

Related U.S. Application Data
(63) Continuation-in-part of application No. 11/801,191, filed on May 9, 2007.
(60) Provisional application No. 61/402,567, filed on Sep. 1, 2010.
(60) Provisional application No. 60/848,278, filed on Sep. 29, 2006.

(51) Int. Cl. A61B 17/70; A61B 17/56

(52) U.S. Cl. 606/249; 606/86 A

(57) ABSTRACT

A side-loading interbody or intervertebral spacer device for placement in an interspinous space between a pair of adjacent spinous processes includes opposed abutment portions sized and shaped for engaging adjacent spinal processes and an arcuate rim. A through-channel is disposed between the opposed abutment portions. A tool configured and arranged for releasably engaging and installing said intervertebral spacer.

### CROSS REFERENCE TO RELATED APPLICATIONS

This application claims the benefit of U.S. Provisional Pat. App. Ser. No. 61/402,567, filed Sep. 1, 2010, the disclosure of which is incorporated by reference herein. This application is also a continuation-in-part of U.S. patent application Ser. No. 11/801,191, filed May 9, 2007, which claims the benefit of U.S. Provisional Application No. 60/848,278 filed Sep. 29, 2006, and is now U.S. Pat. No. ______, both of the disclosures of which are incorporated by reference herein.

### BACKGROUND OF THE INVENTION

The present application is directed to an interbody device for implantation between a pair of adjacent spinous processes for the purpose of providing dynamic support between the vertebrae, and more particularly, to such an intervertebral implant device that is implanted by a lateral minimally invasive method.

In the human spine, the pad or disc between vertebrae can become damaged and deteriorate due to injury, disease or other disorders. Upon such an occurrence, the discs may narrow or flatten, resulting in painful mechanical instability. With particular reference to the lower back, when the disc deteriorates, narrowing and bulging of the disc occurs, causing the two vertebrae that are separated by the disc to move toward one another. This may cause entrapment of nerve roots and resulting pain to the patient.

In an attempt to relieve such lower back pain, intervertebral implants have been designed that include a spacer inserted between the spinous processes. It is known to hold such spacers by ties or bands wrapped around adjacent spinous processes. Such implants advantageously support and limit the movement of the vertebrae treated and yet are not permanently fixed to the vertebral bone, thus avoiding loosening and rigidity issues prevalent in the more permanent bone fixing systems. The non-rigid, removable fixation provided by interspinous process spacers is particularly advantageous, for example, for younger patients needing to manage pain during initial forms of degenerative intervertebral lumbar disc disease, and in some older patients with spinal stenosis and/or degenerative spondylolisthesis.

However, one of the drawbacks to such interspinous spacers is that implantation of the spacer between a pair of adjacent spinous processes requires making an incision centrally along the spine followed by detachment or resection of the supraspinous ligament from at least the two adjacent spinous processes and also resection of the interspinous ligament between the two spinous processes. Detachment and resection of ligaments is not desirable as it is invasive to the patient and extends healing time. In particular, the supraspinous ligament is thicker and broader in the lumbar than in the thoracic region, making such a procedure even more undesirable in the lower back region. It is thus desirable to develop interspinous process spacers and methods of implantation that are less invasive to the patient, preferably such spacers and methods of use that do not require detachment or removal of the supraspinous and interspinous ligaments and that can be inserted laterally from only one side.

### SUMMARY OF THE INVENTION

In a first embodiment, a side-loading interbody or intervertebral spacer device for placement in an interspinous space between a pair of adjacent spinous processes by only one lateral side of the spine is provided. The spacer includes a longitudinal axis that extends from a leading end to a trailing end; a superior-inferior axis that extends from a first side to an second side and is perpendicular to the longitudinal axis; a pair of abutment portions that are located on opposed sides of the longitudinal axis, asymmetric with respect to the longitudinal axis, symmetric with respect to the superior-inferior axis, associated with the first and second sides, and sized and shaped for engaging adjacent superior and inferior spinal processes; a through-channel disposed between the opposed abutment portions and extending the longitudinal axis; and a band receivable in the through-channel.

In a further embodiment, the longitudinal axis is adapted for extending laterally with respect to the adjacent superior and inferior spinal processes after implantation.

In a further embodiment, the superior-inferior axis is adapted for extending superiorly to inferiorly with respect to the adjacent superior and inferior spinal processes after implantation.

In a further embodiment, the pair of abutment portions includes a superior abutment portion and an inferior abutment portion.

In a further embodiment, the superior abutment portion is adapted for snugly engaging the superior spinal process; and the inferior abutment portion is adapted for snugly engaging the inferior spinal process.

In a further embodiment, each of the abutment portions includes a longitudinal portion joined with a perpendicular portion.

In a further embodiment, the longitudinal portion runs parallel with the longitudinal axis; and the perpendicular portion runs parallel to the superior-inferior axis.

In a further embodiment, each longitudinal portion has a cross-section including at least a portion of a shape selected from the group consisting of circles, ellipses, ovals, super ellipses, squovals, squircles, and D-shapes; wherein the cross-section is taken simultaneously perpendicular to the longitudinal axis and parallel to the superior-inferior axis.

In a further embodiment, each of the perpendicular portions includes a perimeter portion including shape selected from the group consisting of partial circles, partial ellipses, partial ovals, partial super ellipses, partial squovals, partial squircles, and D-shapes.

In a further embodiment, an angle defined by the longitudinal and perpendicular portions of a first of the abutment portion is an acute angle; and an angle defined by the longitudinal and perpendicular portions of a second of the abutment portion is obtuse angle; wherein the angles are measured relative to the longitudinal axis and within a plane defined by the longitudinal and superior-inferior axes.

In a further embodiment, the spacer includes a contact portion sized and shaped for engaging the spinous processes; and a non-contact portion.

In a further embodiment, the contact portion includes a curvate surface running parallel with the longitudinal axis and a planar surface extending outwardly therefrom.

In a further embodiment, the non-contact portion includes a length sufficient for extending beyond an engaged pair of spinous processes.

In a further embodiment, the contact portion includes a first portion running parallel with the longitudinal axis and a second portion running substantially parallel with the superior-inferior axis.

In a further embodiment, the non-contact portion includes a pair of spaced legs.

In a further embodiment, the inner legs are spaced a distance at least slightly larger than a width of the band.

In a further embodiment, each leg includes an inner leg surface.

In a further embodiment, the inner leg surfaces run parallel to the longitudinal axis.

In a further embodiment, the inner leg surfaces are joined by a connecting surface.

In a further embodiment, the connecting surface includes a width slightly greater than a width of the band.

In a further embodiment, the connecting surface includes a first through-channel opening adapted to receive the band therethrough.

In a further embodiment, the inner surfaces and the connecting surface define a recessed portion.

In a further embodiment, the trailing end includes a trailing surface spaced from the abutment portions.

In a further embodiment, the trailing surface has a planar surface and a curvate surface.

In a further embodiment, the device includes a connecting portion joining the trailing surface with the abutment portions.

In a further embodiment, the connecting portion includes cross-section with at least a part of a shape selected from the group consisting of circles, ellipses, ovals, super ellipses, squovals, squircles, and D-shapes; wherein the cross-section is taken perpendicular to the longitudinal axis and parallel with the superior-inferior axis.

In a further embodiment, the trailing end includes a band guide portion running parallel with the superior-inferior axis, the band guide portion being adapted for guiding a band therein.

In a further embodiment, the band guide portion includes a recessed surface with a width slightly greater than a width of the band, wherein the width of the recessed surface is measured perpendicular to the superior-inferior axis.

In a further embodiment, the band guide portion includes a second through-channel opening adapted to receive the band therein.

In a further embodiment, the trailing end includes a planar portion running parallel with the superior-inferior axis; and a curvate portion extending outwardly from the longitudinal axis.

In a further embodiment, the trailing end includes a band guide portion sized and shaped for receiving the band; wherein the band guide portion run parallel with the superior-inferior axis; and wherein the band guide portion is in communication with the through-channel, so as to receive the band from the through-channel.

In a further embodiment, the band guide portion includes a pair of co-linear band-engagement surfaces.

In a further embodiment, the band-engagement surfaces are planar.

In a further embodiment, the through-channel includes a second opening associated with the band guide portion.

In a further embodiment, the second opening is substantially rectangular.

In a further embodiment, the band guide portion includes a pair of walls joined by a band-engagement surface; wherein the walls are spaced a distance associated with a width of the band.

In a further embodiment, the walls run parallel to the superior-inferior axis.

In a further embodiment, the device includes an indicia associated with a device installation orientation.

In a further embodiment, the device includes a third side; a fourth side; and an anterior-posterior axis extending from the third side to the fourth side, wherein the anterior-posterior axis is perpendicular to both the longitudinal and superior-inferior axes.

In a further embodiment, the third and fourth sides are planar.

In a further embodiment, each of the third and fourth sides includes a generally T-shaped perimeter region.

In a further embodiment, each of the third and fourth sides includes an opening associated with a tool-engagement bore.

In a further embodiment, each of the third and fourth sides a longitudinal portion running parallel with the longitudinal axis and a perpendicular portion running parallel with superior-inferior axis.

In a further embodiment, the device includes a tool-engagement structure.

In a further embodiment, the device further includes anterior-posterior axis and the tool-engagement structure includes a pair of bores spaced along the anterior-posterior axis.

In a further embodiment, the pair of bores are adapted for engaging an engagement structure of a tool.

In a further embodiment, the tool-engagement structure includes a bore sized and shaped for engaging a tool.

In a further embodiment, the bore includes a pair of bores, the pair of bores being coaxial with an anterior-posterior axis extending from a third side to a fourth side and spaced on opposed sides of the longitudinal axis.

In a further embodiment, the device is asymmetrical with respect to the longitudinal axis and symmetrical with respect to an anterior-posterior axis.

In a further embodiment, the band comprises a pair of bands receivable in the through-channel.

In a further embodiment, the through-channel includes first and second inner wall running parallel with the longitudinal axis and being spaced a distance slightly greater than a width of the band; third and fourth inner walls running parallel with the longitudinal axis and being joined by the first and second inner walls; wherein the through-channel includes a substantially cross-section measured perpendicular to the longitudinal axis.

In a further embodiment, the device further includes an implantation tool adapted for releasably mating with a tool-engagement portion of the device so as to implant the device between the pair of spinous processes.

In a second embodiment, a device for placement between a pair of spinous processes is provided. The device includes a cap portion having a superior-inferior axis of orientation, a trailing end with a trailing surface and a band engagement surface, each of the surfaces running parallel with the superior-inferior axis, and first and second engagement portions; a stem portion extending perpendicularly from the cap portion and including a leading surface, third and fourth engagement portions, the third engagement portion being joined with the first engagement portion, and the fourth engagement portion being joined with the second engagement portion; a band-receiving channel extending from the cap trailing end to the stem leading surface, and being sized and shaped to receive a band therethrough; and a band sized and shaped to extend through the band-receiving channel and around a first spinous process in cooperative abutment with the first abutment portion.

In a further embodiment, the device includes a second band sized and shaped to extend through the band-receiving channel and around a second spinous process in cooperative abutment with a second surface of the second abutment portion.

In a further embodiment, the stem is sized and shaped for distraction of the first and second spinous processes.

In a further embodiment, the device further includes a tool-engagement structure.

In a further embodiment, the device further includes an indicia associated with an orientation of device installation.

In a third embodiment, an installation tool for installing an interspinous process spacer is provided. The installation tool includes an elongate handle extending from a top end to a bottom end along a longitudinal axis; and an engagement subassembly extending perpendicularly from the bottom end of the handle with respect to the longitudinal axis, and being adapted for engaging and implant an interspinous process spacer between a pair of adjacent spinous processes of a spine.

In a further embodiment, the engagement subassembly includes a peg adapted for engaging a tool engagement structure of the interspinous process spacer.

In a further embodiment, the peg is sized and shaped for releasably mating with a bore of the tool engagement structure.

In a further embodiment, the peg includes a second peg sized and shaped for releasably mating with another bore of the tool engagement structure.

In a further embodiment, the peg includes a curvate surface and a circular cross-section, wherein the cross-section is taken perpendicular with the longitudinal axis.

In a further embodiment, the peg extends downwardly and parallel with the handle longitudinal axis.

In a further embodiment, the peg includes a first peg that is immobile with respect to the handle, and a mobile second peg, wherein the first and second pegs are spaced and co-linear.

In a further embodiment, the handle includes a movable handle portion slidingly received by a stationary handle portion; and the engagement subassembly includes a foot associated with the stationary handle portion and including a downwardly extending first pin, and an arm associated with the movable handle portion, the arm being spaced a distance from the foot and including a downwardly extending second pin; and wherein sliding the movable handle portion with respect to the stationary handle portion changes the distance between the foot and the arm.

In a further embodiment, the first and second pins are spaced along a pin central axis running parallel with the longitudinal axis of the handle.

In a further embodiment, the second pin is adapted for moving upward and downward with respect to the first pin.

In a further embodiment, the tool includes a first position associated with the first and second pins being spaced by a first distance; and a second position associated with the first and second pins being spaced by a second distance; wherein the second distance is associated with engaging a tool engagement structure of the interspinous process spacer.

In a further embodiment, the tool includes an engagement position associated with an open configuration of the spacer-engagement subassembly; and a non-engagement position associated with a closed configuration of the spacer-engagement subassembly.

In a fourth embodiment, an installation tool for installing a laterally-loading interspinous process spacer is provided, including an outer arm having a longitudinally extending channel; an inner arm slidingly engaged within the channel; and a pin sized and shaped for releasably mating with a pin-receiving channel of an interspinous process spacer.

In a further embodiment, the spacer-engaging subassembly includes a first position associated with non-engagement of said spacer and a second position associated with releasably engagement of said spacer.

In a further embodiment, the tool further includes an interspinous spaced adapted for releasable mating with the tool and lateral implantation between a pair of adjacent spinous processes.

### BRIEF DESCRIPTION OF THE DRAWINGS

The drawings constitute a part of this specification and include exemplary embodiments of the present invention and illustrate various objects and features thereof.

FIG. 1 is an enlarged perspective view of an interspinous spacer of the invention.

FIG. 2 is a rear elevational view of the spacer of FIG. 1.

FIG. 3 is a top plan view of the spacer of FIG. 1.

FIG. 4 is a cross-sectional view, taken along the line 4-4 of FIG. 3.

FIG. 5 is a cross-sectional view taken along the line 5-5 of FIG. 2.

FIG. 6 is an enlarged partial perspective and generally schematic view of a patient's spine showing an early stage of performing an incision in a process according to the invention.

FIG. 7 is an enlarged partial perspective and generally schematic view similar to FIG. 6, showing a preparation stage of a process according to the invention subsequent to the stage shown in FIG. 6.

FIG. 8 is an enlarged perspective view of an interspinous trial spacer.

FIG. 9 is a front elevational view of the trial spacer of FIG. 8.

FIG. 10 is a cross-sectional view taken along the line 10-10 of FIG. 9.

FIG. 11 is a top plan view of the trial spacer of FIG. 8.

FIG. 12 is an enlarged exploded partial perspective view of the trial spacer of FIG. 8 shown with a snap-on tool.

FIG. 13 is an enlarged partial side elevational view of the spacer and tool of FIG. 12 with portions broken away to show detail thereof.

FIG. 14 is an enlarged and partial view, similar to FIG. 7, showing insertion of the trial spacer and tool of FIGS. 12 and 13 according to a method of the invention.

FIG. 15 is an enlarged and partial view, similar to FIG. 14, showing a subsequent step of a method according to the invention wherein the trial spacer is inserted into an interspinous space as a test to determine an appropriately sized spacer of FIG. 1.

FIG. 16 is a perspective view showing a pair of tools for use in processes of the invention.

FIG. 17 is a an enlarged and partial view, similar to FIG. 15, showing the incision and a portion of the patient's spine, and further showing an early stage of implantation of a band according to a method of the invention with one of the tools shown in FIG. 16.

FIG. 18 is an enlarged and partial top plan view of the patient's spine, band and tool shown in FIG. 17, further illustrating the lateral nature of the process step of FIG. 17, with portions of the spine that are not exposed by the incision being shown in phantom.

FIG. 19 is an enlarged and partial top plan view, similar to FIG. 18, showing a later stage in the band implantation process.

FIG. 20 is an enlarged and partial top plan view, similar to FIGS. 18 and 19, showing implantation of a second band according to a method of the invention.

FIG. 21 is an enlarged and partial top plan view, similar to FIG. 20, showing a later stage in the implantation of the second band.

FIG. 22 is an enlarged and partial perspective view, similar to FIGS. 14, 15 and 17, showing both of the bands being implanted.

FIG. 23 is an enlarged and partial perspective view, similar to FIG. 22, showing a subsequent method step of threading of the spacer of FIG. 1 onto the bands.

FIG. 24 is an enlarged and partial perspective view, similar to FIG. 23, showing a subsequent method step of positioning the bands.

FIG. 25 is an enlarged and partial perspective view, similar to FIG. 24, showing a subsequent method step of inserting the spacer of FIG. 1 into an interspinous space.

FIG. 26 is an enlarged and partial top plan view, similar to FIGS. 18-21, showing a method step subsequent to the step shown in FIG. 25 wherein the bands are being secured about the spinous process by a pair of band holding and tensioning tools.

FIG. 27 is an enlarged and partial top plan view, similar to FIG. 26 showing the bands finally secured about the spinous process.

FIG. 28 is an enlarged and partial perspective view showing the band tightening process shown in FIGS. 26 and 27.

FIG. 29 is an enlarged and partial top plan view, similar to FIG. 18, showing an early stage in a process according to the invention of implanting at least two spacers of FIG. 1 wherein a first spacer is being threaded.

FIG. 30 is an enlarged and partial top plan view, similar to FIG. 29 showing a subsequent step of implantation of a band for the second spacer.

FIG. 31 is an enlarged and partial top plan view, similar to FIG. 30, showing a subsequent step of threading the second spacer.

FIG. 32 is an enlarged and partial top plan view, similar to FIG. 31, showing a subsequent step of band attachment and tightening.

FIG. 33 is an enlarged and partial perspective view showing the two spacers of FIG. 32 fully implanted with the bands tightened.

FIG. 34 is an enlarged perspective view of a second embodiment of an interspinous spacer of the invention.

FIG. 35 is a front elevational view of the spacer of FIG. 34.

FIG. 36 is a top plan view of the spacer of FIG. 34.

FIG. 37 is a cross-sectional view, taken along the line 37-37 of FIG. 36.

FIG. 38 is a cross-sectional view taken along the line 38-38 of FIG. 35, and also showing the spacer with portions of the spinous process and a first band attaching the spacer to the spinous process.

FIG. 39 is an enlarged partial perspective and generally schematic view of a patient's spine showing an early stage of performing an incision in a process according to the invention.

FIG. 40 is an enlarged partial perspective and generally schematic view similar to FIG. 39, showing a preparation stage of a process according to the invention subsequent to the stage shown in FIG. 39.

FIG. 41 is an enlarged and partial view, similar to FIG. 40, showing insertion of a trial spacer and the tool of FIGS. 12 and 13 according to a method of the invention wherein the trial spacer is inserted in an interspinous space as a test to determine an appropriately sized spacer of FIG. 34.

FIG. 42 is a perspective view showing a pair of tools for use in processes of the invention.

FIG. 43 is a an enlarged and partial view, similar to FIG. 41, showing the incision and a portion of the patient's spine, and further showing an early stage of implantation of a band according to a method of the invention with one of the tools shown in FIG. 42.

FIG. 44 is an enlarged and partial top plan view of the patient's spine, band and tool shown in FIG. 43, further illustrating the lateral nature of the process step of FIG. 43, with portions of the spine that are not exposed by the incision being shown in phantom.

FIG. 45 is an enlarged and partial top plan view, similar to FIG. 44, showing a later stage in the band implantation process.

FIG. 46 is an enlarged and partial top plan view, similar to FIGS. 43 and 44, showing subsequent implantation of a second band according to a method of the invention.

FIG. 47 is an enlarged and partial top plan view, similar to FIG. 46, showing a later stage in the implantation of the second band.

FIG. 48 is an enlarged and partial perspective view showing both of the bands being implanted prior to threading of the spacer of FIG. 34.

FIG. 49 is an enlarged and partial perspective view, similar to FIG. 48, showing a subsequent method step of threading of the spacer of FIG. 34 onto the bands.

FIG. 50 is an enlarged and partial perspective view, similar to FIG. 49, showing a subsequent method step of inserting the spacer of FIG. 34 into an interspinous space.

FIG. 51 is an enlarged and partial top plan view, showing a method step subsequent to the step shown in FIG. 50 wherein the bands are being secured about the spinous process using a pair of band holding tools.

FIG. 52 is an enlarged and partial top plan view, similar to FIG. 51 showing the bands finally secured about the spinous process.

FIG. 53 is an enlarged and partial perspective view showing two spacers of FIG. 34 fully implanted with the bands tightened according to a process of the invention.

FIG. 54 is a top perspective view of a third embodiment of an interspinous spacer of the invention; the arrow CR indicates a cranial orientation or direction after implantation.

FIG. 55 is a top plan view of the spacer of FIG. 54.

FIG. 56 is a perspective view of the bottom of the spacer of FIG. 54.

FIG. 57 is a bottom plan view of the spacer of FIG. 54.

FIG. 58 is a rear plan view of the spacer of FIG. 54.

FIG. 59 is a side elevational view of the spacer of FIG. 54.

FIG. 60 is a front plan view of the spacer of FIG. 54.

FIG. 61 is a cross-sectional view of the spacer of FIG. 58, taken along the line 61-61 of FIG. 58, and also showing the spacer with portions of a pair of adjacent spinous processes and a first band attaching the spacer to the superior spinous process.

FIG. 62 is a cross-sectional view of the spacer of FIG. 58, taken along the line 62-62 of FIG. 58.

FIG. 63 is a side perspective view of an installation tool of the invention, for implanting the side-loading intervertebral process spacer of FIG. 54.

FIG. 64 is a side plan of the installation tool of FIG. 63.

FIG. 65 is a front plan view of the installation tool of FIG. 63.

FIG. 66 is a bottom plan view of the installation tool of FIG. 63.

FIG. 67 is a partial rear plan of the installation tool of FIG. 63.

FIG. 68 is a top plan view of the installation tool of FIG. 63.

FIG. 69 is a partial side plan view of the installation tool of FIG. 63 in a first position, with the spacer of FIG. 62.

FIG. 70 is a partial side plan view of the installation tool of FIG. 63 in a second position, with the spacer of FIG. 62.

FIG. 71 is a partial view of an incision and a portion of the patient's spine, and further showing an early stage of implantation of a band.

FIG. 72 is a partial top plan view of the patient's spine, the band and the tool shown in FIG. 71, further illustrating the lateral nature of the process step of FIG. 71, with portions of the spine that are not exposed by the incision being shown in phantom.

FIG. 73 is a partial top plan view, similar to FIG. 72, showing a later stage in the band implantation process.

FIG. 74 is a partial top plan view, similar to FIG. 73, showing subsequent implantation of a second band according to a method of the invention.

FIG. 75 is a partial top plan view, similar to FIG. 74, showing a later stage in the implantation of the second band.

FIG. 76 is a partial perspective view showing both of the bands being implanted prior to threading of the spacer of FIG. 54

FIG. 77 is a partial perspective view, similar to FIG. 76, showing a subsequent method step of threading of the spacer of FIG. 54 onto the bands.

FIG. 78 is a partial perspective view showing a method step subsequent to the step shown in FIG. 77, wherein the spacer of FIG. 54 is being threaded on the bands toward an interspinous space.

FIG. 79 is a partial perspective view, similar to FIG. 78, showing a method step of inserting the spacer of FIG. 54 into an interspinous space, using the installation tool of FIG. 63.

FIG. 80 is a partial top plan view, with portions broken way to shown the detail there of, showing a method step subsequent to the step shown in FIG. 79, with the spacer of FIG. 54 shown in cross-section, the cross-section being taken on lines 61-61 of FIG. 58, wherein the bands are being secured about the spinous processes using a pair of band holding tools.

FIG. 81 is a partial perspective view showing later step of the process of the invention, wherein the spacer is in the interspinous space and with the bands tightened, one band having been clipped to a final length and the other band being adjusted.

FIG. 82 is a partial top plan view, with portions broken away to shown the detail there of, similar to FIG. 80, with the spacer of FIG. 54 shown in cross-section, showing the bands secured about the spinous processes and clipped to a final length.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

It is also noted that any reference to the words top, bottom, up and down, and the like, in this application refers to the alignment shown in the various drawings, as well as the normal connotations applied to such devices, and is not intended to restrict positioning of the spacers in actual use. It is also noted that reference to words such as front, back, anterior and posterior used in this application also refer to the alignment shown in the various drawings, and in particular, when possible, with reference to the human spine and human body, but also is not intended to restrict positioning of the spacers in actual use.

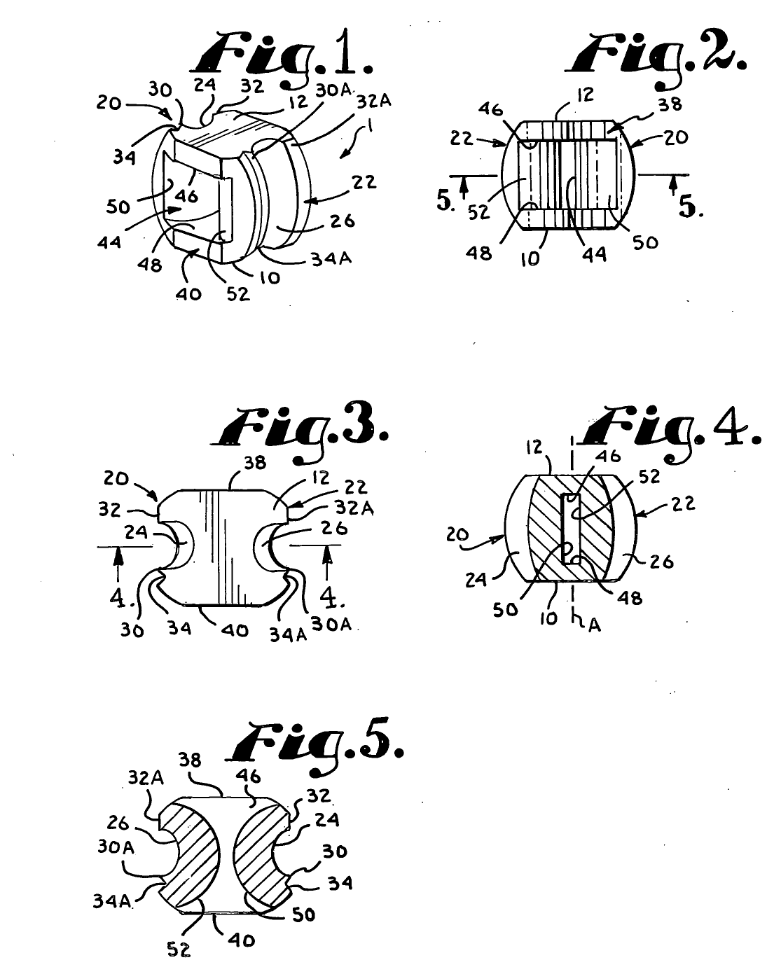

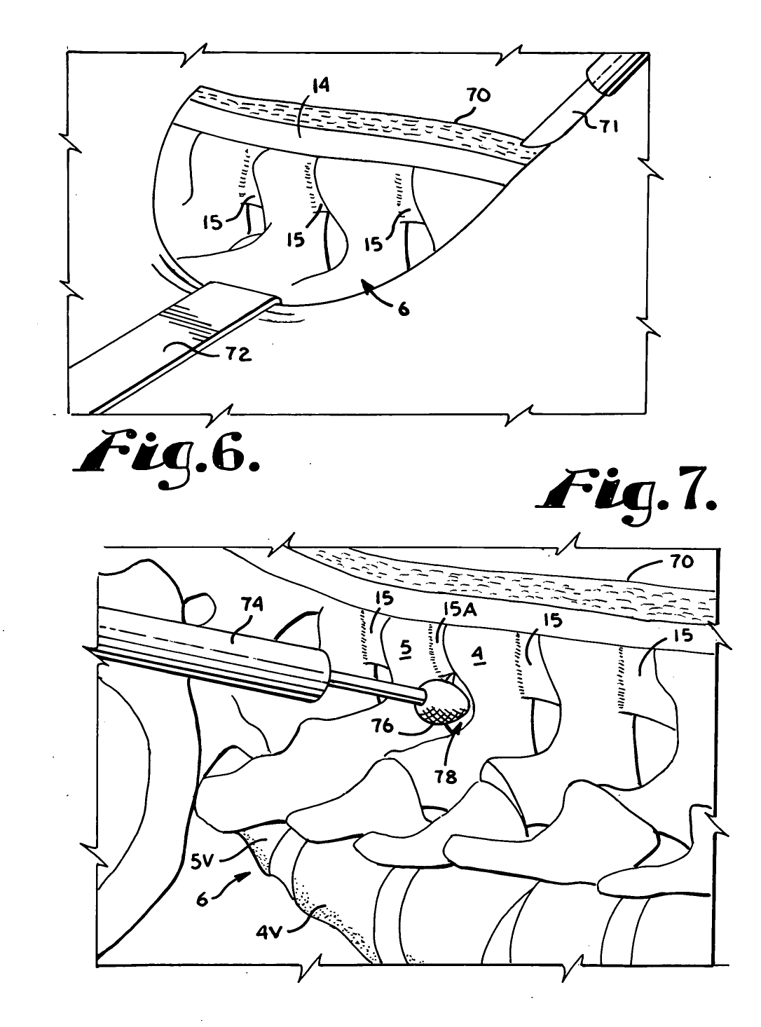

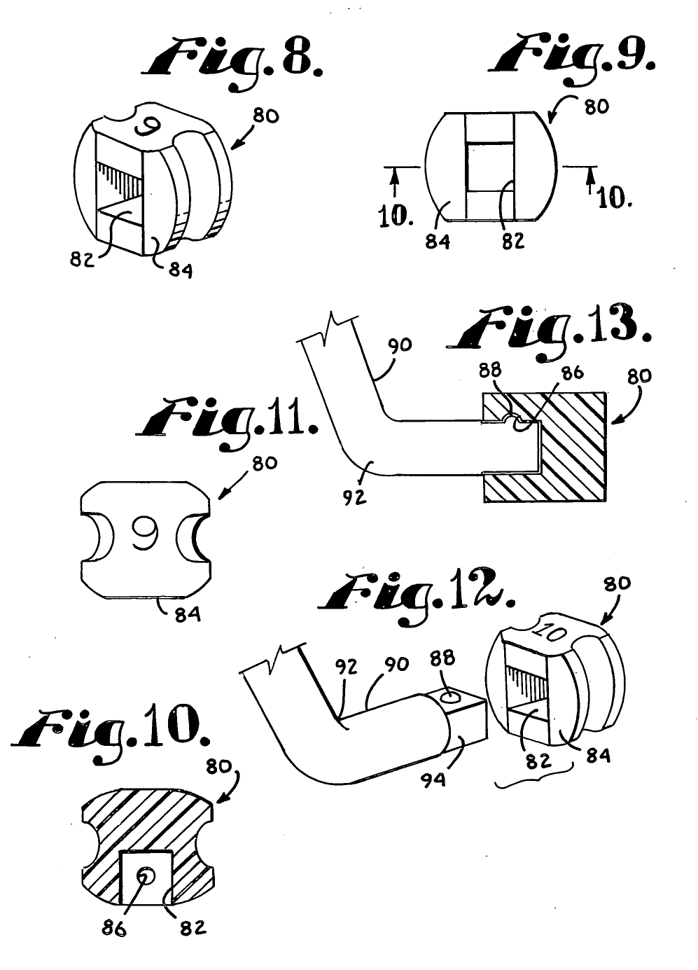

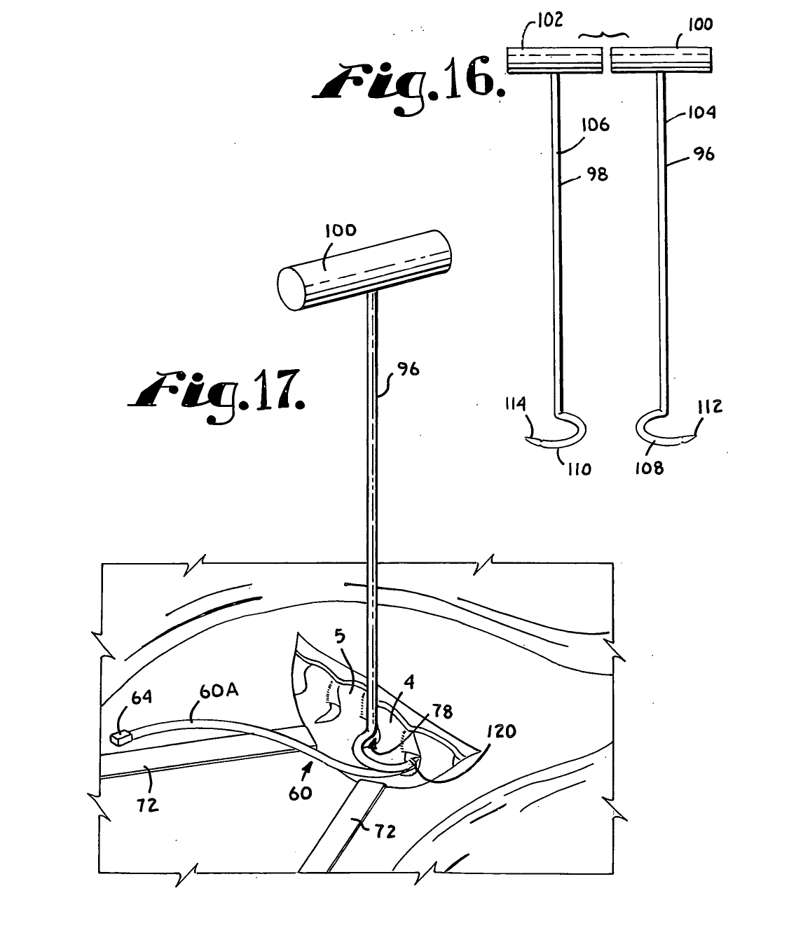

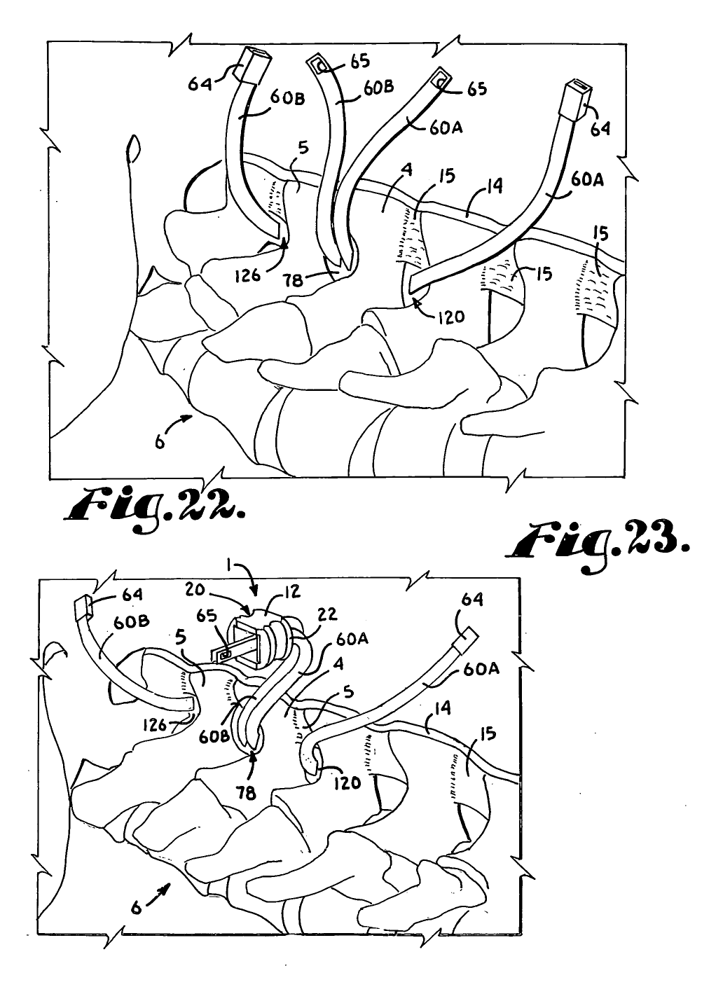

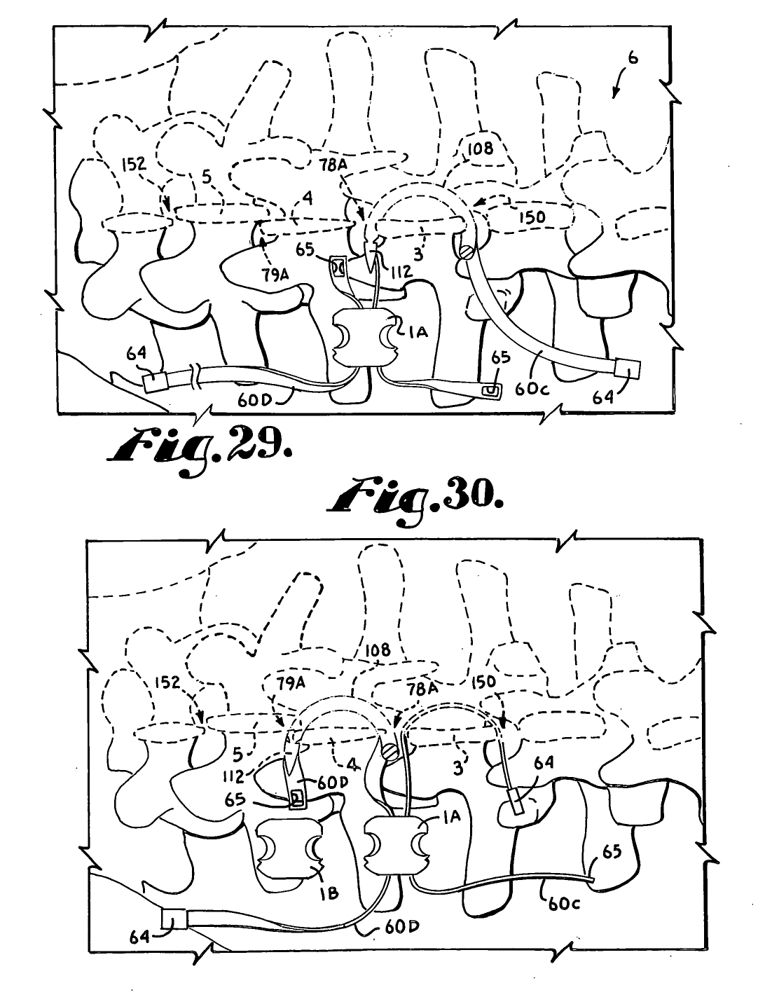

With reference to FIGS. 1-28, the reference numeral 1 generally designates an spinous process spacer device of the present invention. The device 1 is used to maintain proper spacing between a pair of spinous processes 4 and 5 of a human spine, preferably a lower portion thereof, generally 6. The device 1 is preferably constructed from a single, unitary blank or molded, strong structure. The device 1 may be rigid or somewhat elastic and may be made from metals, metal alloys, plastics and/or composites. For example, the device 1 may be molded or machined from polymer or plastic materials such as polyetheretherketone (PEEK), silastics and polyurethanes. Devices 1 according to the invention are made in incremental sizes so that a desired size of device 1 is implanted for cooperating with a particular patient's spine 6.

In particular, the illustrated device 1 has a substantially flat anterior surface 10 for facing toward vertebrae 4V and 5V of the lower spinal portion 6 and a substantially flat posterior surface 12 opposite the surface 10 for facing away from the vertebrae and toward the supraspinous ligament 14 and a portion of interspinous ligament 15 that is disposed between the pair of spinous processes 4 and 5, the surface 12 being identical or substantially similar to the surface 10. The surfaces 10 and 12 are disposed in substantially parallel planes; however, such surfaces could be non-parallel.

The device 1 further includes an inferior or lower saddle-like abutment face or portion, generally 20, and an opposite substantially identical or similar superior or upper abutment face or portion, generally 22. The portions or faces 20 and 22 are arcuate having a substantially convex outer profile, curving substantially uniformly outwardly from the opposed surfaces 10 and 12. Formed in faces 20 and 22 are respective substantially uniform and centrally located U-shaped and arcuate furrows or channels 24 and 26 sized and shaped for engagement with the respective spinous processes 5 and 4 prepared for receiving the device 1 according to a method of the invention as will be described in greater detail below. The channels 24 and 26 are non-linear, having a substantially convex outer profile running from the surface 10 to the surface 12. The channels 24 and 26 are each defined in part by a pair of rims or ridges; the channel 24 being bounded by the rims or ridges 30 and 32 and the channel 26 being bounded by the rims or ridges 30A and 32A. Formed in the rims 30 and 30A are tool receiving grooves 34 and 34A, respectively. Although grooves 34 and 34A are shown that run from the surface 10 to the surface 12, it is foreseen that other types of tool receiving formations may be used including but not limited to notches, apertures and the like.

The rims 32 and 32A are adjacent to a lateral insertion or leading face or portion, generally 38 that is disposed opposite a lateral trailing face or portion, generally 40. The grooved rims 30 and 30A are disposed near the face 40. Both faces 38 and 40 are preferably beveled or rounded near the respective rims 30, 30A and 32 and 32A to aid in inserting the device 1 between the spinous processes 4 and 5. A band receiving channel 44 extends through the device 1 between the lateral faces 38 and 40. The channel is further defined by a pair of opposed substantially flat walls 46 and 48 and a pair of arcuate walls 50 and 52. The walls 46 and 48 are substantially parallel to one another and to the anterior and posterior surfaces 10 and 12. The walls 50 and 52 curve towards one another. As illustrated in FIG. 4, at a smallest opening in an interior of the device 1, the walls defining the channel 44 form an opening having a substantially rectangular cross-section, a length of the rectangular cross-section extending along a substantially central axis A. The channel 44 then widens in a direction toward the face 38 and also substantially the same in an opposite direction toward the face 44 as best illustrated in FIGS. 1, 2 and 5.

One or more devices 1 according to the invention are implantable between pairs of spinous processes attaching to adjacent spinous processes utilizing bands, ties or tapes, generally 60. The device 1 and attached bands provide for non-rigid stabilization without permanent fixation in the vertebral bone. The device 1 also relieves low-back pain due to disc pathology and symptoms from spinal stenosis caused by degenerative disc disease, spinal arthritis and instability, being useful as an initial substitute to pedicle screw fixation for dynamic spinal stabilization, total disc prostheses and spinal fusion methods.

Specifically, a pair of identical bands or ribbons, 60A and 60B are illustrated in FIGS. 17-28. Each of the bands 60A and 60B is elongate and substantially flat, preferably having limited elasticity to allow for sufficient tightening about a spinous process and a portion of the device 1. The bands may be formed of a monofilament or woven, braided or otherwise formed, and prepared from a variety of materials including plastics, such as thermoplastic polymer resins, silastics including polyesters, for example, polyethylene terephthalate (PET). The bands 60A and 60B are sized and shaped to be received in the through-channel 44 and fit snugly against both a spinous process and one of the interior curved walls 50 or 52 of the device 1. Each band 60A and 60B includes an attached self-locking, anti-slip fixing structure or buckle 64 on one end thereof and an aperture or eyelet 65 formed near an opposite end thereof. The locking structures 64 are known in the art, and for example, include a channel for extending the band 60 therethrough to form a loop about a portion of the device 1 and thereafter tighten the loop as will be described more fully below. Within the buckle channel, the locking structure or buckle 64 includes, for example, reverse angle teeth, hooks or pressure structure that prohibits the band 60 from feeding back through the buckle 64 once threaded into and through the buckle and tensioned, as illustrated in FIG. 26.

In use, an incision 70 is first made with a cutting tool 71 and held open with a tool 72 near the spinous processes 4 and 5. As illustrated in FIG. 6, according to methods of the invention, the incision 70 is advantageously located in the midline or somewhat laterally to the spine, on one side only; and it is not necessary to detach or section the supraspinous ligament 14 or the interspinous ligament 15 in advance of implantation of the device 1, thus providing a more minimally invasive procedure with muscle dissection and gentle soft tissue retraction limited to just one side.

With reference to FIG. 7, a cutting or shaving tool 74 having a rounded working surface 76 is then used to prepare an implantation site by inserting the tool 74 into the incision and utilizing the working surface 76 to remove small portions of the spinous processes 4 and 5 and a lower portion 15A of the interspinous ligament 15, if necessary, adjacent to an interspinous space, generally 78, into which the device 1 will eventually be implanted. The tool working surface 76 is rotated and/or otherwise maneuvered until a desired shape of interspinous space 78 results that substantially conforms to a desired form for engagement with the faces 20 and 22 of the device 1.

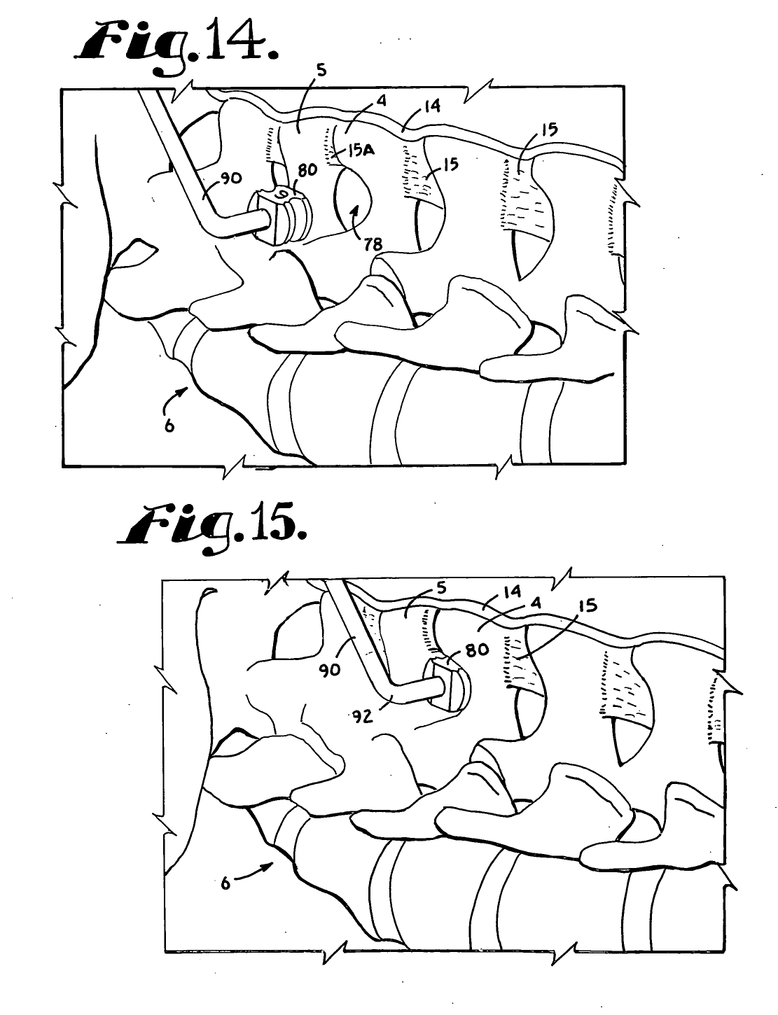

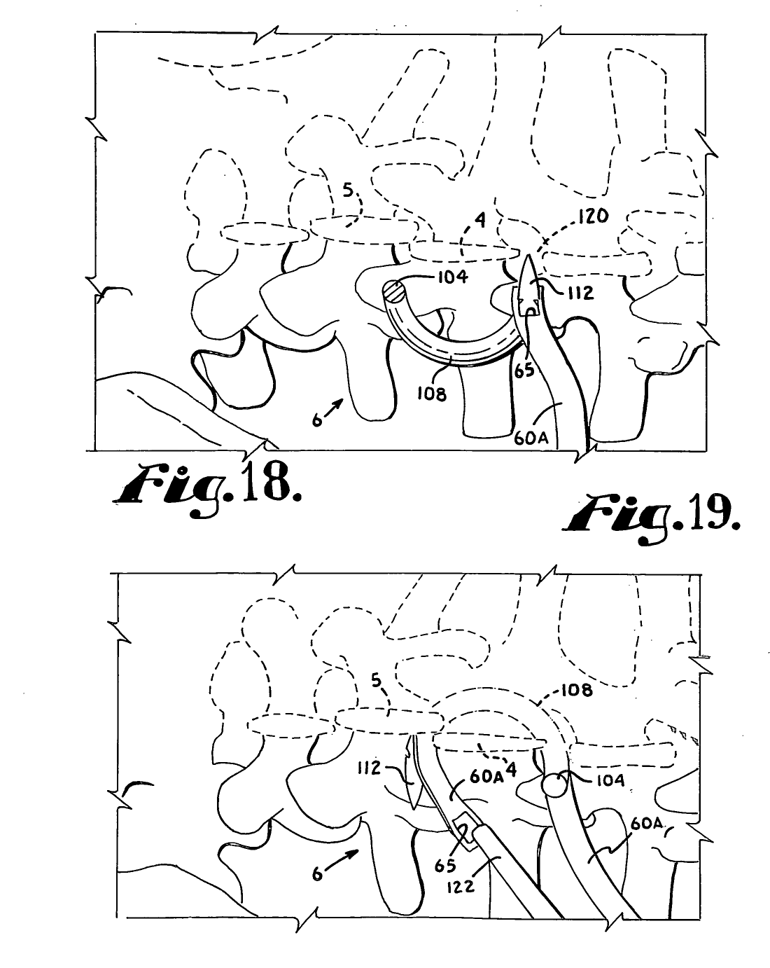

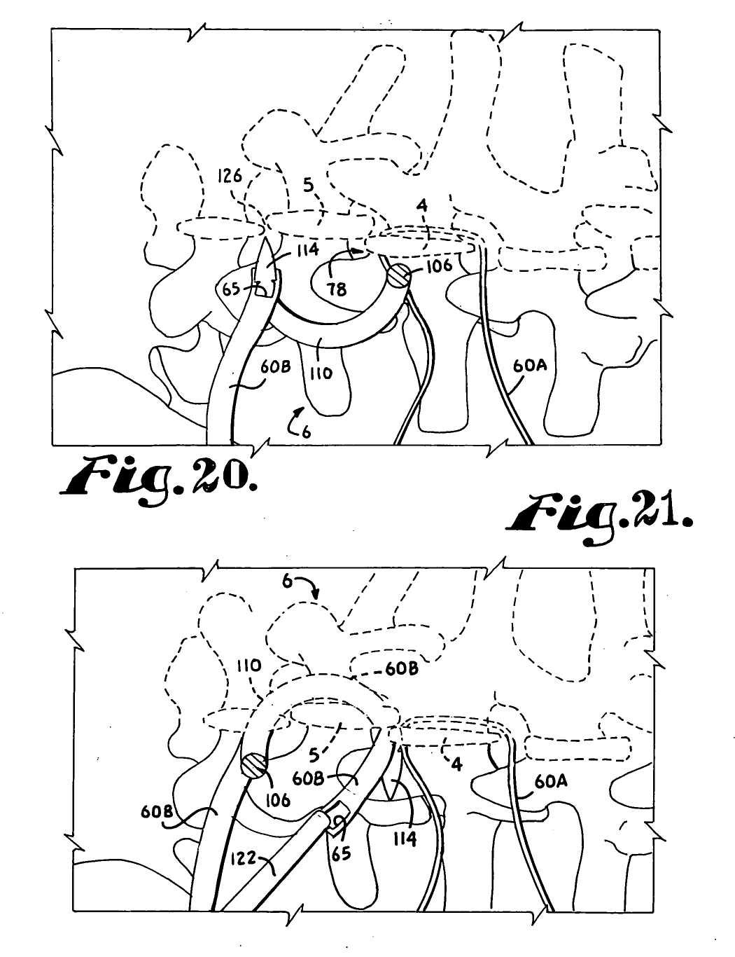

With particular reference to FIGS. 8-15, in order to determine an adequate size of device 1 to fit with a particular patient's spinous process spacing and anatomy, one or more trial spacers 80 are tested. Each trial spacer 80 is substantially similar to a device 1 with the exception that in lieu of a curved through-channel 44, the spacer 80 includes an aperture 82 formed in a lateral face 84. The surfaces forming the aperture 82 include a further recess 86 for receiving a knob 88 of an insertion tool 90. The tool 90 is elongate and includes a bend 92 to allow for lateral insertion of the trial spacer 80 into the interspinous space 78. A substantially block-shaped end portion 94 of the tool 90 is received in the aperture 82 with the knob 88 snapping or otherwise engaging into the recess 86 as illustrated in FIGS. 12 and 13. Then, as illustrated in FIGS. 14 and 15, the trial spacer 80 is carefully inserted into the interspinous space 78 and tested for correct fit. As illustrated in FIG. 8, each size of trial spacer may be identified with a numeral imprinted thereon to indicate size, and each available size of spacer 80 may be tested until a correct fit is achieved. A spacer device 1 of the invention is then chosen that is of the same size as the chosen trial spacer 80. Each trial spacer 80 has an identical aperture 82 such that the same insertion tool 90 may be used with each sized trial spacer 80.

With particular reference to FIGS. 16-22, after the trial spacer 80 is removed, the bands 60A and 60B are each implanted at the site 78. With reference to FIG. 16, the bands are implanted utilizing the band insertion tools 96 and 98. The tools 96 and 98 include respective handles 100 and 102; respective elongate shafts 104 and 106; respective curved hook portions 108 and 110 disposed perpendicular to the respective shafts 104 and 106; and respective pointed tips 112 and 114 terminating the respective hook portions 108 and 110. The band insertion tools 96 and 98 are identical with the exception of a direction of curvature of the hook portions 108 and 110. The hook portion 108 curves in a counterclockwise direction from the shaft 104 substantially forming a half circle and the hook portion 110 curves in a clockwise direction from the shaft 106 substantially forming a half circle.

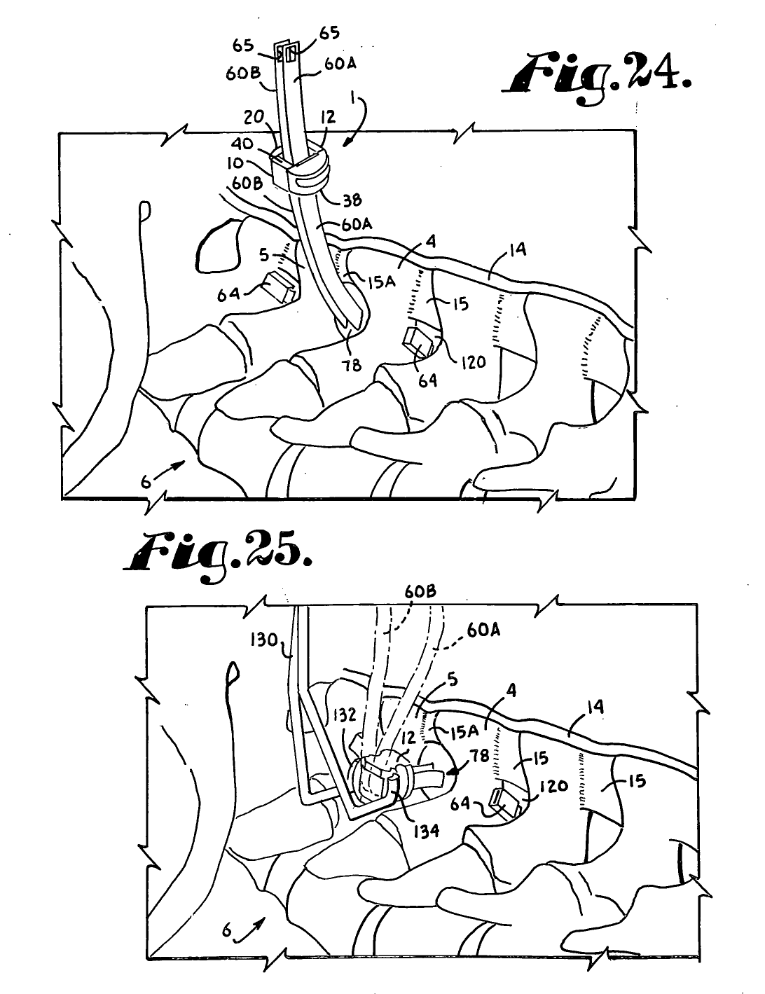

With reference to FIGS. 17-19, the tool 96 is utilized to thread the band 60A about the spinous process 4 and through the space or site 78. The pointed tip 112 is inserted into the eyelet 65 of the band 60A and the tip 112 with the threaded band 60A are inserted into a space 120 located adjacent to the spinous process 4 on the other side of the prepared space 78. The handle 100 of the tool 96 is then rotated, rotating the shaft 104 and in particular the semi-circular hook portion 108 in a counterclockwise direction about the spinous process 4, threading the band 60A through the interspinous space 78. With reference to FIG. 19, a grasping tool, such as a forceps 122 is used to seize and hold the band 60A, pulling the band 60A and turning the band 60A such that a flat surface thereof is disposed about the spinous process 4 as illustrated in FIG. 20. The handle 100 of the tool 96 is then rotated in a clockwise manner to turn the hook portion 108 back out of the interspinous space 78 and then out of the space 120.

With reference to FIGS. 20-21, the band 60B is implanted in a method similar to that described above with respect to the band 60A. The pointed tip 114 is inserted into the eyelet 65 of the band 60B and the tip 114 with the threaded band 60B are inserted into a space 126 located adjacent to the spinous process 5 on the other side of the prepared space 78. The handle 102 of the tool 98 is then rotated, rotating the shaft 106 and in particular the semi-circular hook portion 110 in a clockwise direction about the spinous process 5, threading the band 60B through the interspinous space 78. With reference to FIG. 21, a grasping tool, such as the forceps 122 is used to seize and hold the band 60B, pulling the band 60B and turning the band 60B such that a flat surface thereof is disposed about the spinous process 5 as illustrated in FIG. 22. The handle 102 of the tool 98 is then rotated in a counterclockwise manner to turn the hook portion 110 out of the interspinous space 78 and then the space 126.

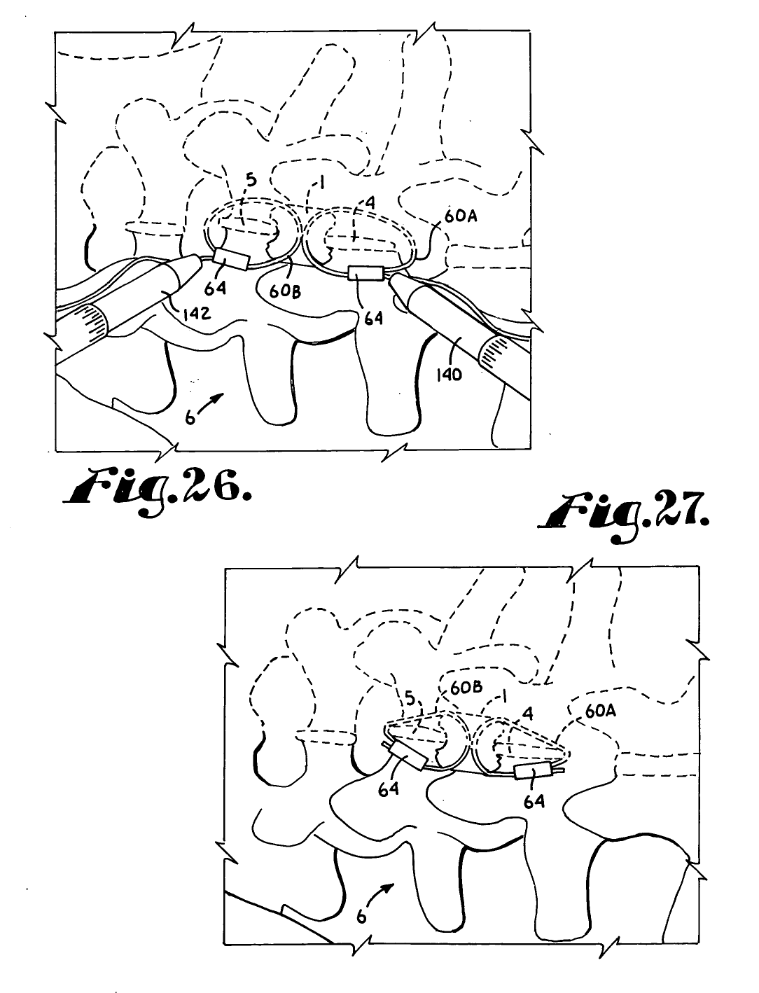

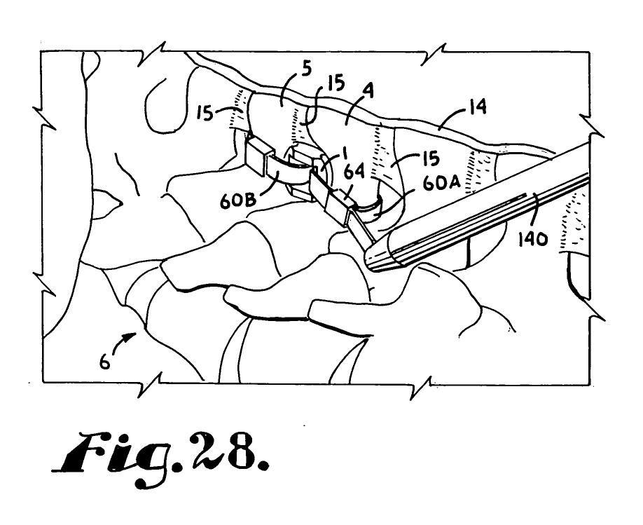

With further reference to FIG. 22 and with reference to FIG. 23, the bands 60A and 60B are pulled to a desired position with the eyelets 65 of the bands aligned after which both bands 60A and 60B are threaded into the through-channel 44 of the device 1 at the lateral insertion face 38 and toward the trailing face 40, the flat surfaces of the bands being adjacent to the curved walls 50 and 52 defining the channel 44. With reference to FIG. 24, as the bands 60A and 60B are pulled through the channel 44, the fixing structures or buckles 64 are pulled into position adjacent the interspinous spaces 120 and 126. With reference to FIG. 25, a spacer holding tool 130 is used to hold and insert the spacer device 1 into the site 78 utilizing prongs 132 and 134 that engage respective tool grooves 34 and 34A on the device 1.

With reference to FIGS. 26-28, once the device 1 is inserted into the site 78 with the faces 20 and 22 engaging the spinous processes 5 and 4 respectively, a pair of band grasping tools 140 and 142 are utilized to thread the bands 60A and 60B through respective buckles 64 thereon and tighten the bands, the buckles 64 locking the bands snugly in place about the spinous processes 4 and 5 and surfaces of the spacer device 1. The bands 60A and 60B are then trimmed.

Removal of the device 1, if necessary, includes the following steps: cutting the bands 60A and 60B; removing the band portions from the interspinous spaces 120 and 126; and then removing the device 1 from the interspinous space 78.

With reference to FIGS. 29-33, two or more devices 1 according to the invention may be implanted according to a method of the invention into adjacent interspinous spaces. The implantation procedure is similar to that described previously herein with respect to a single device 1. The example illustrated in FIGS. 29-33 and described herein includes two devices 1A and 1B that are identical to the device 1 previously described herein and also bands 60C, 60D and 60E identical to the bands 60, 60A and 60B previously described herein. For this example, the same human spine 6 is illustrated, along with spinous processes 3, 4, and 5. Although not shown in the drawings, similar to what was previously described herein, interspinous process spaces 78A and 79A are first prepared utilizing the tool 74 shown in FIG. 7. Trial spacers 80 are then inserted into the spaces to determine a correctly sized spacer 1A and a correctly sized spacer 1B as illustrated in FIGS. 14 and 15 and previously described herein.

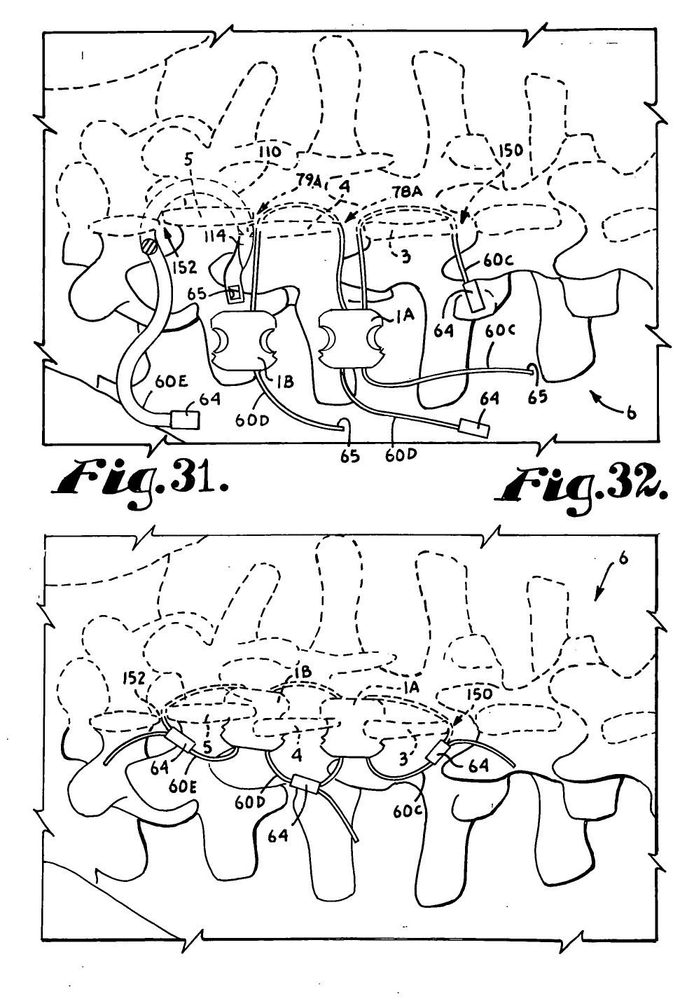

With reference to FIG. 29, after removal of the trial spacer 80, the tool 96 is utilized to thread the band 60C about the spinous process 3 and through the space or site 78A. The pointed tip 112 is inserted into the eyelet 65 of the band 60C and the tip 112 with the threaded band 60C are inserted into a space 150 located adjacent to the spinous process 3 on the other side of the prepared space 78A. The handle 100 of the tool 96 is then rotated, rotating the shaft 104 and in particular the semi-circular hook portion 108 in a counterclockwise direction about the spinous process 3, threading the band 60C through the interspinous space 78A. Similar to what is shown in FIG. 19, a grasping tool, such as a forceps 122 is used to seize and hold the band 60C, pulling the band 60C and turning the band 60C such that a flat surface thereof is disposed about the spinous process 3 as illustrated in FIG. 30. The handle 100 of the tool 96 is then rotated in a clockwise manner to turn the hook portion 108 back out of the interspinous space 78A and then out of the space 150. The band 60C is then threaded through the channel 44 of the device 1A by inserting the eyelet 65 end into the channel 44 at the leading face 38 and out of the trailing face 40 as illustrated in FIG. 30.

Also with reference to FIG. 30, the tool 96 is again utilized to thread the band 60D about the spinous process 4 and through the space or site 79A. However, before insertion, the band 60D is threaded through the device 1A by inserting the end of the band 60D having the eyelet 65 into the device 1A at the trailing face 40, through the channel 44 and out of the leading face 38. The pointed tip 112 of the tool is then inserted into the eyelet 65 of the band 60D and the tip 112 with the threaded band 60D are inserted into the prepared space 78A. The handle 100 of the tool 96 is then rotated, rotating the shaft 104 and in particular the semi-circular hook portion 108 in a counterclockwise direction about the spinous process 4, threading the band 60D through the interspinous space 79A. Similar to what is shown in FIG. 19, a grasping tool, such as a forceps 122 is used to seize and hold the band 60D, pulling the band 60D and turning the band 60D such that a flat surface thereof is disposed about the spinous process 4 as illustrated in FIG. 31. The handle 100 of the tool 96 is then rotated in a clockwise manner to turn the hook portion 108 back out of the interspinous space 79A and then out of the space 78A. The band 60D is then threaded through the channel 44 of the device 1B by inserting the eyelet 65 end into the channel 44 at the leading face 38 and out of the trailing face 40 as illustrated in FIG. 31.

Also with reference to FIG. 31, the band 60E is implanted in a method similar to that described above with respect to the bands 60C and D, but with the tool 98. The pointed tip 114 is inserted into the eyelet 65 of the band 60E and the tip 114 with the threaded band 60E are inserted into a space 152 located adjacent to the spinous process 5 on the other side of the prepared space 79A. The handle 102 of the tool 98 is then rotated, rotating the shaft 106 and in particular the semi-circular hook portion 110 in a clockwise direction about the spinous process 5, threading the band 60E through the interspinous space 79A. A grasping tool, such as the forceps 122 is used to seize and hold the band 60E, pulling the band 60E and turning the band 60E such that a flat surface thereof is disposed about the spinous process 5. The handle 102 of the tool 98 is then rotated in a counterclockwise manner to turn the hook portion 110 out of the interspinous space 79A and then the space 152. The band 60E is then threaded through the channel 44 of the device 1B by inserting the eyelet 65 end into the channel 44 at the leading face 38 and out of the trailing face 40.

With reference to FIG. 32, the bands 60C, 60D and 60E are pulled through the channels 44 of the devices 1A and 1B, and the fixing structures or buckles 64 of each of the bands 60C and 60E are pulled into position adjacent the interspinous spaces 150 and 152. The spacer holding tool 130 is used to hold and insert the spacer device 1A into the site 78A utilizing prongs 132 and 134 that engage respective tool grooves 34 and 34A on the device 1A. The spacer holding tool 130 is also used to hold and insert the spacer device 1B into the site 79A utilizing prongs 132 and 134 that engage respective tool grooves 34 and 34A on the device 1B.

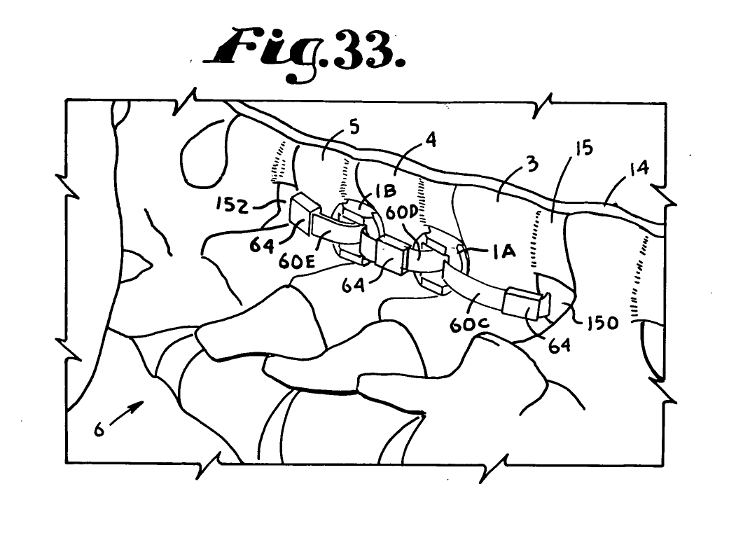

With reference to FIGS. 32-33, band grasping tools 140 and 142 are then utilized to thread the bands 60C, 60D and 60E through respective buckles 64 thereon and tighten the bands, the buckles 64 locking the bands snugly in place about the respective spinous processes 3, 4 and 5 and surfaces of the spacer devices 1A and 1B. The bands 60C, 60D and 60E are then trimmed.

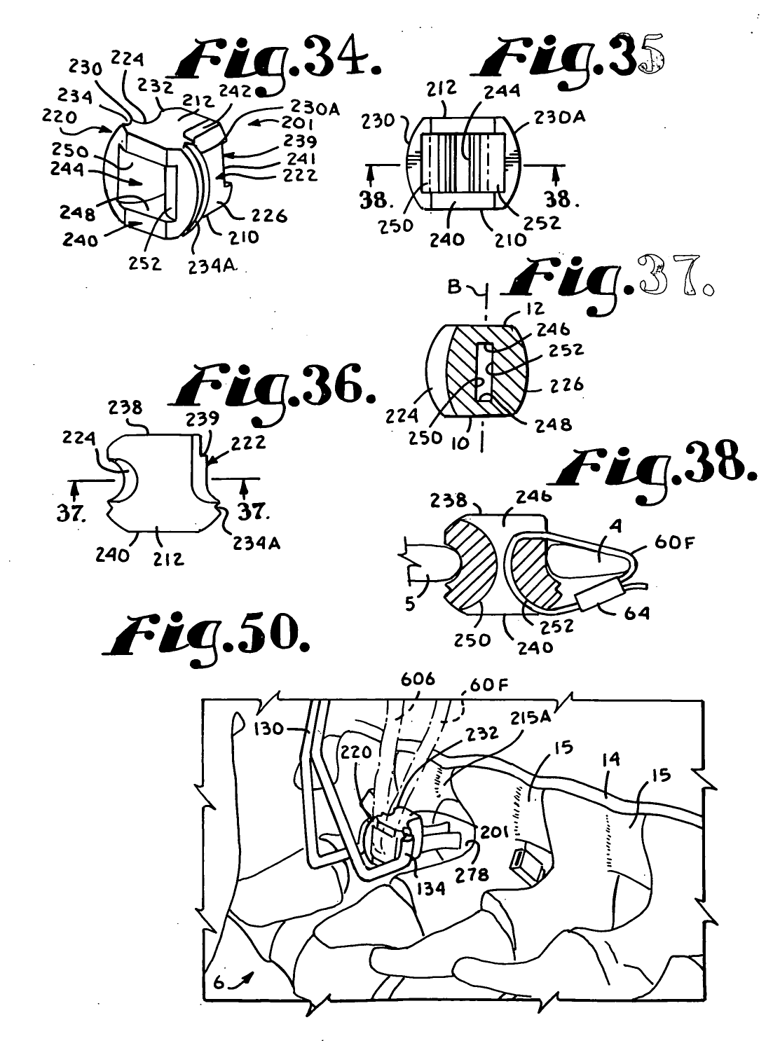

With reference to FIGS. 34-53, the reference numeral 201 generally designates an alternative embodiment of an interspinous process spacer device of the present invention. The device 201 is also used to maintain proper spacing between a pair of spinous processes 4 and 5 of a human spine, preferably a lower portion thereof, generally 6. The device 201 is preferably constructed from a single, unitary blank or molded, strong structure. The device 201 may be rigid or somewhat elastic and may be made from metals, metal alloys, plastics and/or composites. For example, the device 201 may be molded or machined from a plastic material such as polyetheretherketone (PEEK). Devices 201 according to the invention are made in incremental sizes so that a desired size of device 201 is implanted for cooperating with a particular patient's spine 6.

In particular, the illustrated device 201 has a substantially flat anterior surface 210 for facing toward vertebrae 4V and 5V of the lower spinal portion 6 and a substantially flat posterior surface 212 opposite the surface 210 for facing away from the vertebrae and toward the supraspinous ligament 14 and a portion of interspinous ligament 15 that is disposed between the pair of spinous processes 4 and 5, the surface 212 being identical or substantially similar to the surface 210. The surfaces 210 and 212 are disposed in substantially parallel planes.

The device 201 further includes an inferior or lower saddle-like abutment face or portion, generally 220, and an opposite superior or upper abutment face or portion, generally 222 that is not saddle-like in nature, thus providing an asymmetric device having lateral implantation advantages to be discussed in greater detail below. The portion or face 220 is substantially arcuate and includes an arcuate U-shaped furrow or channel 224 defined in part by arcuate rims 230 and 232. The portion or face 222 includes a slightly arcuate face 226 and an arcuate rim 230A that is similar to the rim 230. Unlike the rim 232, the portion or face 222 does not include a second or leading rim. The absence of such a rim aids in implanting the device 201, wherein the device 201 may be rotated or turned into place between the spinous processes 4 and 5, requiring less preparation and removal of bone, ligament and other body tissue around an interspinous space 278 that is the implantation site for the device 201. In order to provide stability and structure to keep a band 60F and attached device 201 in place with respect to the spinous process 4, a portion of the face 222 and a leading face 238 includes a depression or squared-off notch 239 sized and shaped such that a width of the band 60F fits within the notch 239, the notch further defined by a flat side 241 so that a flat surface of the band 60F fits snugly against the device 201 at the notch 239.

Each of the arcuate portions 224, 226, 230, 232 and 230A have a substantially convex outer profile, curving substantially uniformly outwardly from the opposed surfaces 210 and 212. The U-shaped channel 224 formed in the face 220 is substantially uniform and centrally located and otherwise sized and shaped for engagement with the spinous processes 5. The channel 224 is non-linear, having a substantially convex outer profile running from the surface 210 to the surface 212. Formed in the rims 230 and 230A are tool receiving grooves 234 and 234A, respectively. Although grooves 234 and 234A are shown that run from the surface 210 to the surface 212, it is foreseen that other types of tool receiving formations may be used including but not limited to notches, apertures and the like.

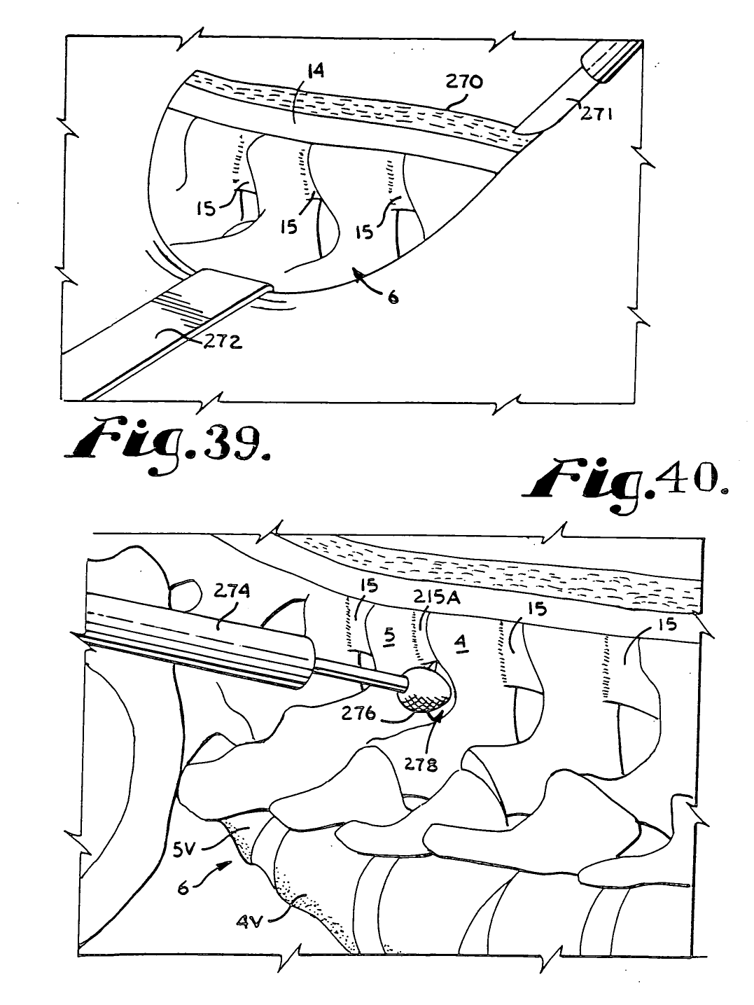

The rim 232 and the notch 239 are each adjacent to the lateral insertion or leading face or portion, generally 238 that is disposed opposite a lateral trailing face or portion, generally 240. The grooved rims 230 and 230A are disposed near the face 240. Both faces 238 and 240 are preferably beveled or rounded to aid in inserting the device 201 between the spinous processes 4 and 5. The surface 212 also includes a bevel 242. A band receiving channel 244 extends through the device 201 between the lateral faces 238 and 240. The channel is further defined by a pair of opposed substantially flat walls 246 and 248 and a pair of arcuate walls 250 and 252. The walls 246 and 248 are substantially parallel to one another and to the anterior and posterior surfaces 210 and 212. The walls 250 and 252 curve towards one another, but are not mirror images as shown in FIG. 38 due to the presence of the notch 239 that guides and controls the location of the band 60F as described above. As illustrated in FIG. 37, at a smallest opening in an interior of the device 201, the walls defining the channel 244 form an opening having a substantially rectangular cross-section, a length of the rectangular cross-section extending along a substantially central axis B. The channel 244 then widens in a direction toward the face 238 and also in an opposite direction toward the face 240 as best illustrated in FIG. 38.

One or more devices 201 according to the invention are implantable between pairs of spinous processes attaching to adjacent spinous processes utilizing bands, ties or tapes, generally 60 as described previous herein and as shown as ties 60F and 60G in the drawing figures. With reference to FIG. 39, in use, an incision 270 is first made with a cutting tool 271 and held open with a tool 272 near the spinous processes 4 and 5. The incision 270 is advantageously located laterally to the spine, on one side only; and it is not necessary to detach or section the supraspinous ligament 14 or the interspinous ligament 15 in advance of implantation of the device 201. Thus providing a minimally invasive procedure.

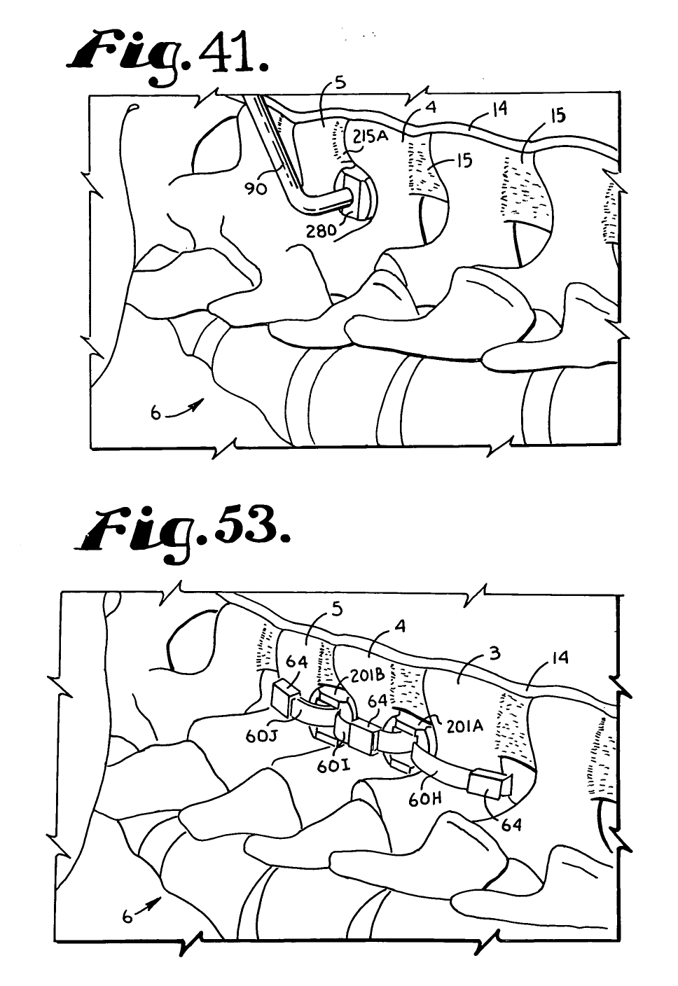

With reference to FIG. 40, a cutting or shaving tool 274 having a rounded working surface 276 is then used to prepare an implantation site by inserting the tool 274 into the incision and utilizing the working surface 276 to remove small portions of the spinous processes 4 and 5 and a portion 215A of the interspinous ligament 15, if necessary, adjacent to an interspinous space, generally 278, into which the device 201 will eventually be implanted. The tool working surface 276 is rotated and/or otherwise maneuvered until a desired shape of interspinous space 278 results that substantially conforms to a desired form for engagement with the faces 220 and 222 of the device 201. As indicated above, as compared to the space 78 required for the device 1, the space 278 may be made slightly smaller, advantageously allowing for the removal of less bone and ligament in view of the asymmetrical geometry of the device 201 wherein a rim 32A is replaced by a notch 239.

With reference to FIG. 41, in order to determine an adequate size of device 201 to fit with a particular patient's interspinous process space, one or more trial spacers 280 are tested. Each trial spacer 280 is substantially similar to a device 201 with the exception that in lieu of a curved through-channel 244, the spacer 280 includes an aperture similar or identical to the aperture 82 previously described herein with respect to the trial spacer 80 and the device 1. Thus the tool 90 cooperates with the spacer 280 as previously described herein to allow for measurement of the space 278 to determine the appropriately sized device 201.

With particular reference to FIGS. 42-47, after the trial spacer 280 is removed, the bands 60F and 60G are each implanted at the site 278. With reference to FIGS. 16 and 42, the bands are implanted utilizing the band insertion tools 96 and 98 previously described herein.

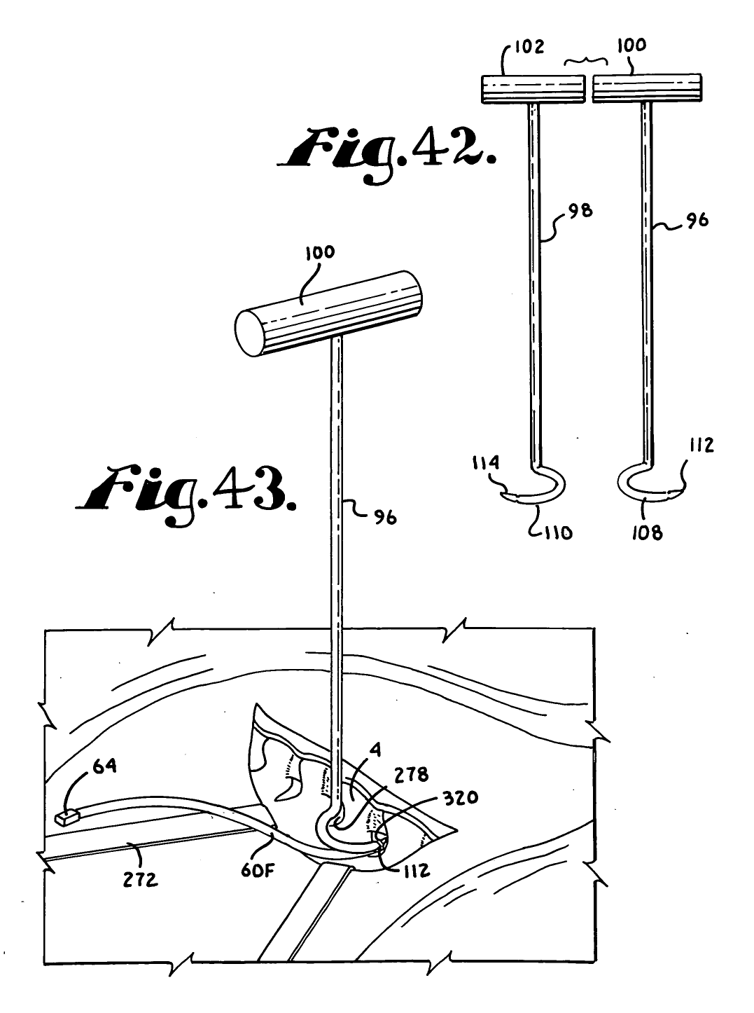

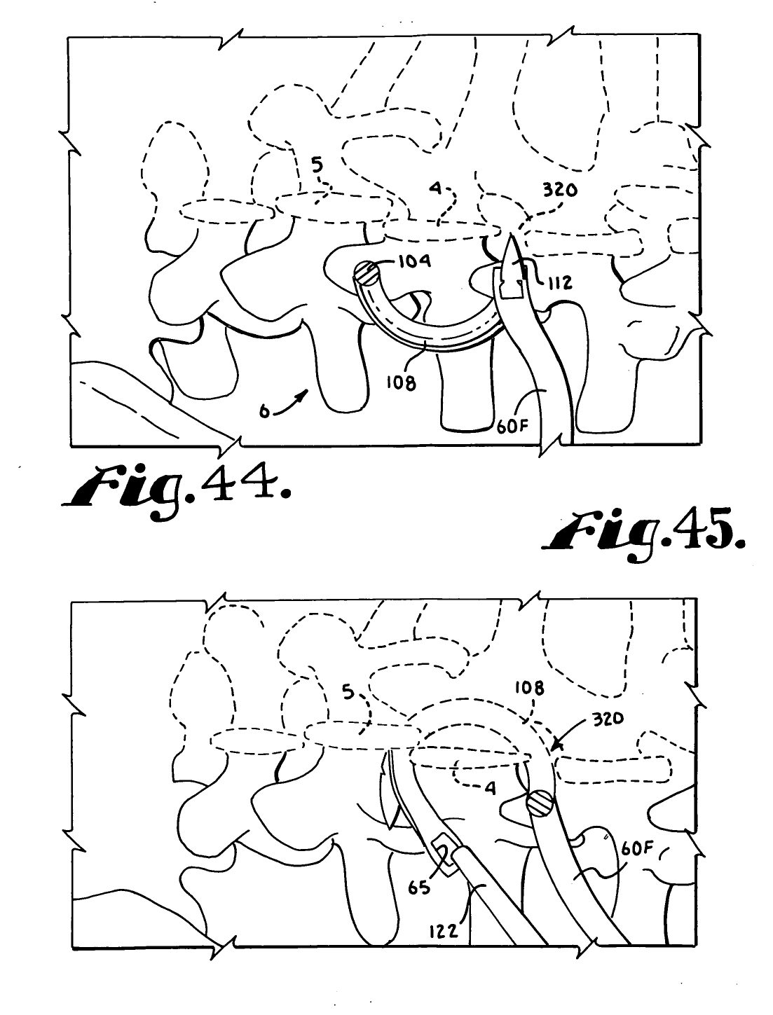

With reference to FIGS. 43-45, the tool 96 is utilized to thread the band 60F about the spinous process 4 and through the space or site 278. The pointed tip 112 is inserted into the eyelet 65 of the band 60F and the tip 112 with the threaded band 60F are inserted into a space 320 located adjacent to the spinous process 4 on the other side of the prepared space 278. The handle 100 of the tool 96 is then rotated, rotating the shaft 104 and in particular the semi-circular hook portion 108 in a counterclockwise direction about the spinous process 4, threading the band 60F through the interspinous space 278. With reference to FIG. 45, a grasping tool, such as the forceps 122 is used to seize and hold the band 60F, pulling the band 60F and turning the band 60F such that a flat surface thereof is disposed about the spinous process 4 as illustrated in FIG. 46. The handle 100 of the tool 96 is then rotated in a clockwise manner to turn the hook portion 108 back out of the interspinous space 278 and then out of the space 320.

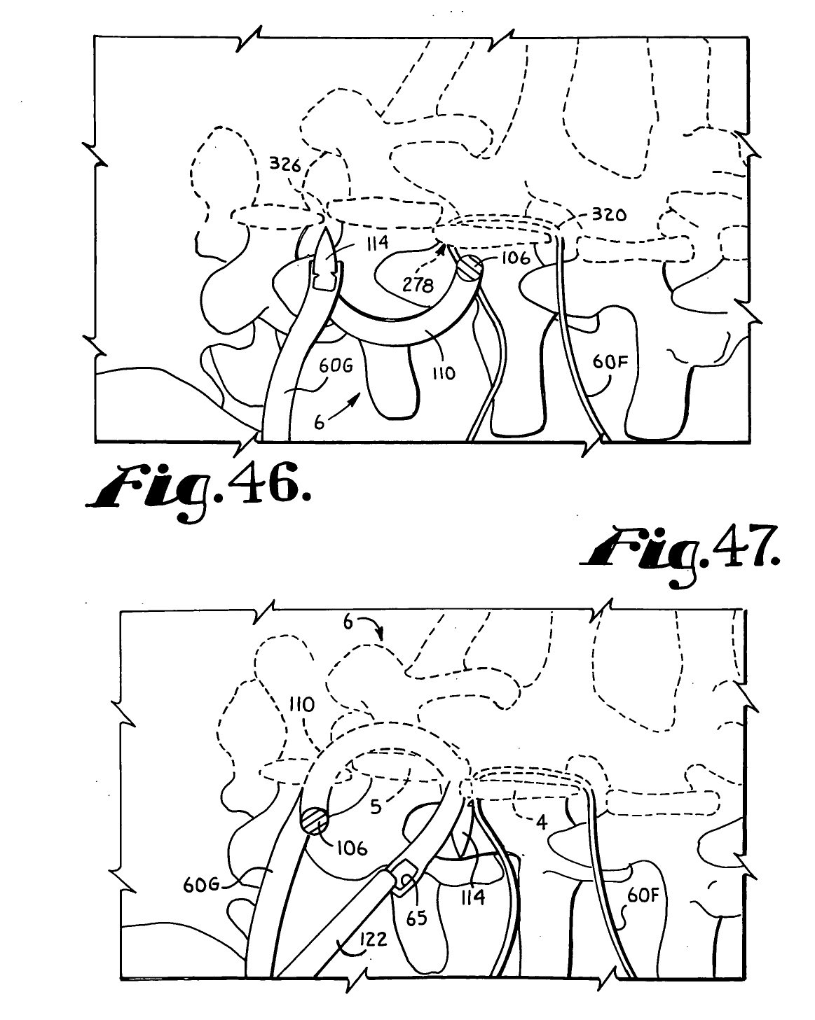

With reference to FIGS. 46-47, the band 60G is implanted in a method similar to that described above with respect to the band 60G. The pointed tip 114 is inserted into the eyelet 65 of the band 60G and the tip 114 with the threaded band 60 GB are inserted into a space 326 located adjacent to the spinous process 5 on the other side of the prepared space 278. The handle 102 of the tool 98 is then rotated, rotating the shaft 106 and in particular the semi-circular hook portion 110 in a clockwise direction about the spinous process 5, threading the band 60G through the interspinous space 278. With reference to FIG. 47, a grasping tool, such as the forceps 122 is used to seize and hold the band 60G, pulling the band 60G and turning the band 60G such that a flat surface thereof is disposed about the spinous process 5 as illustrated in FIGS. 47 and 48. The handle 102 of the tool 98 is then rotated in a counterclockwise manner to turn the hook portion 110 out of the interspinous space 278 and then the space 326.

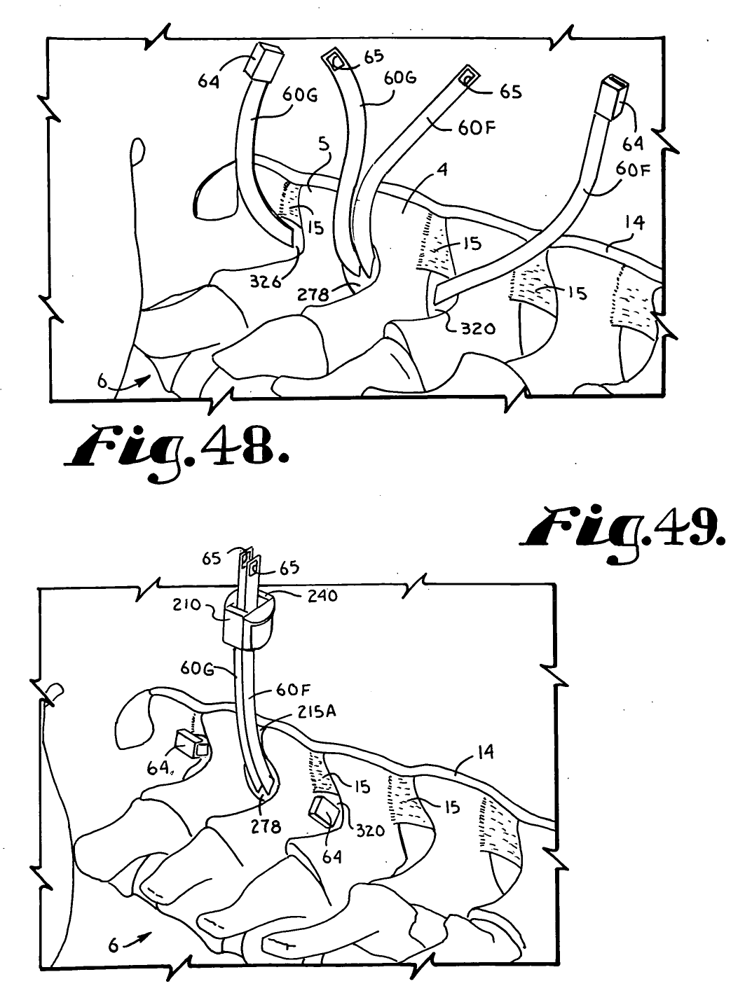

With further reference to FIG. 48 and with reference to FIG. 49, the bands 60F and 60G are pulled to a desired position with the eyelets 65 of the bands aligned after which both bands 60F and 60G are threaded into the through-channel 244 of the device 201 at the lateral insertion face 238 and toward the trailing face 240, the flat surfaces of the bands being adjacent to the curved walls 250 and 252 defining the channel 244. With reference to FIG. 49, as the bands 60F and 60G are pulled through the channel 244, the fixing structures or buckles 64 are pulled into position adjacent the interspinous spaces 320 and 326. With reference to FIG. 50, the spacer holding tool 130 previously described herein is used to hold and insert the spacer device 201 into the site 278 utilizing prongs 232 and 234 that engage respective tool grooves 234 and 234A on the device 201. As discussed above, the device 201 is inserted into the space 278 with the face 220 in the lead, being directed toward the spinous process 5, rather than moving the face 238 directly and evenly between the processes 4 and 5. Once the rim 232 is located in the space 278 near the spinous process 5, the device 201 is rotated into place, with the rim 230A being moved into place next to the spinous process 4. The device 201 is then in place as shown in FIG. 38.

With reference to FIGS. 51-52, once the device 201 is inserted into the site 278 with the faces 220 and 222 engaging the spinous processes 5 and 4 respectively, a pair of band grasping tools 140 and 142 are utilized to thread the bands 60F and 60G through respective buckles 64 thereon and tighten the bands, the buckles 64 locking the bands snugly in place about the spinous processes 4 and 5 and surfaces of the spacer device 201. The bands 60F and 60G are then trimmed.

Removal of the device 201, if necessary, includes the following steps: cutting the bands 60F and 60G; removing the band portions from the interspinous spaces 320 and 326; and then removing the device 201 in a rotating manner from the interspinous space 278, first removing the device at the rim 230A and rotating generally towards the spinous process 5.

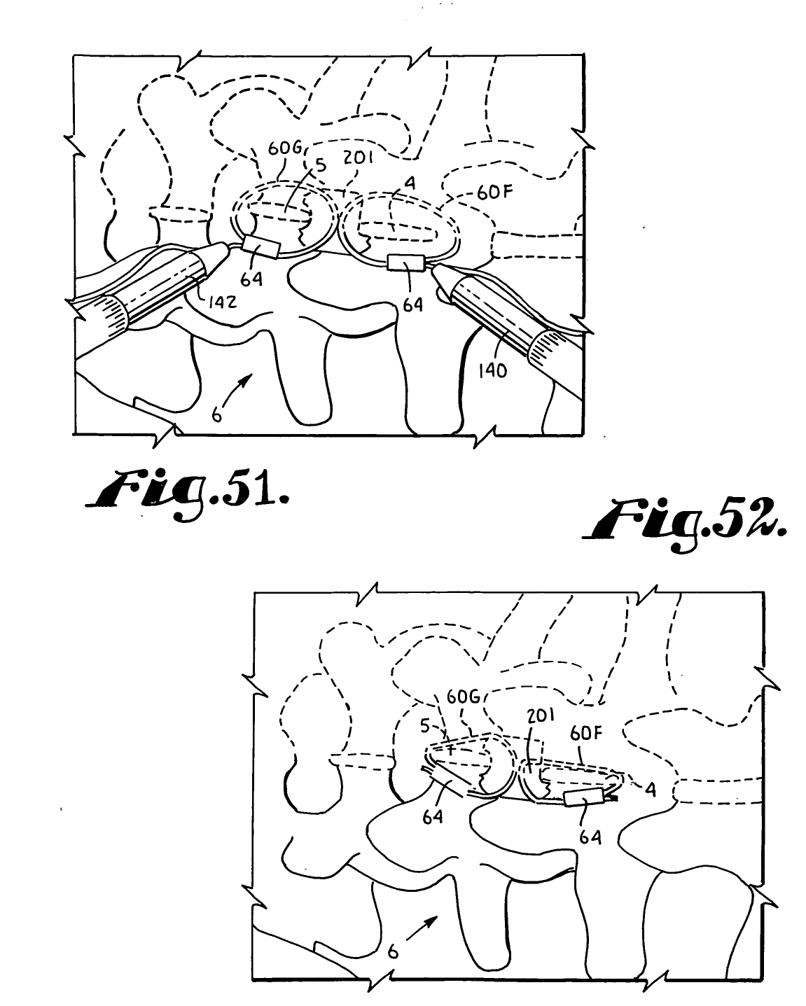

With reference to FIG. 53 a pair of devices 201A and 201B are shown implanted between adjacent spinal processes 3, 4, and 5. The devices 201A and 201B are the same or substantially similar to the device 201 previously described herein. The devices 201A and 201B are implanted according to a method previously described herein with respect to the devices 1A and 1B and illustrated in FIGS. 29-33.

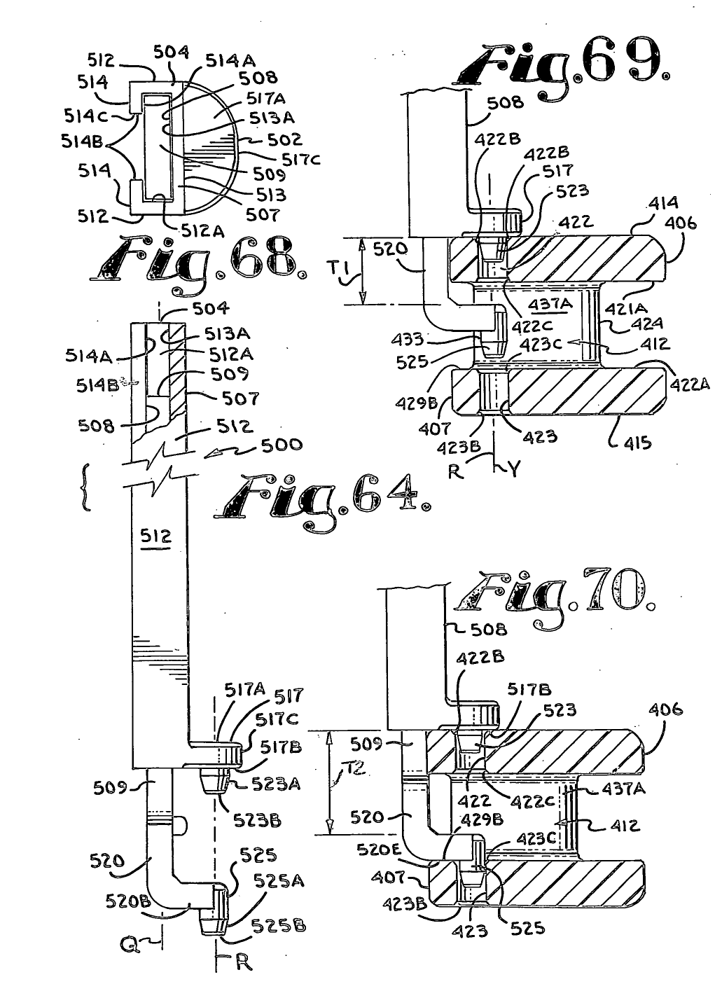

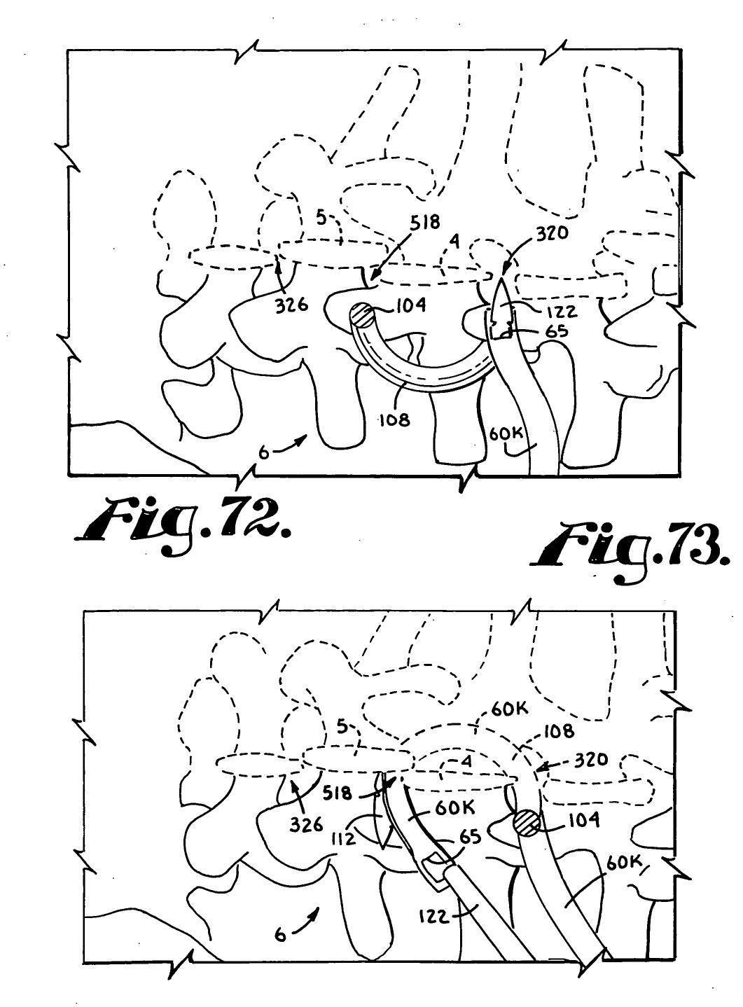

FIGS. 54-82 illustrate another embodiment of an interspinous process spacer device, generally 401, of the present invention. The spacer 401 is also used to maintain proper spacing between a pair of spinous processes, such as but not limited to exemplary spinous processes 4 and 5, of a human spine, preferably a lower portion thereof, generally 6. The spacer 401 is preferably constructed from a single, unitary blank or molded, strong structure. The spacer 401 may be rigid or somewhat elastic and may be made from metals, metal alloys, plastics and/or composites. For example, the spacer 401 may be molded and/or machined from a plastic material such as polyetheretherketone (PEEK). Devices 401 according to the invention are made in incremental sizes so that a desired size of spacer 401 is implanted for cooperating with a particular patient's spine 6.

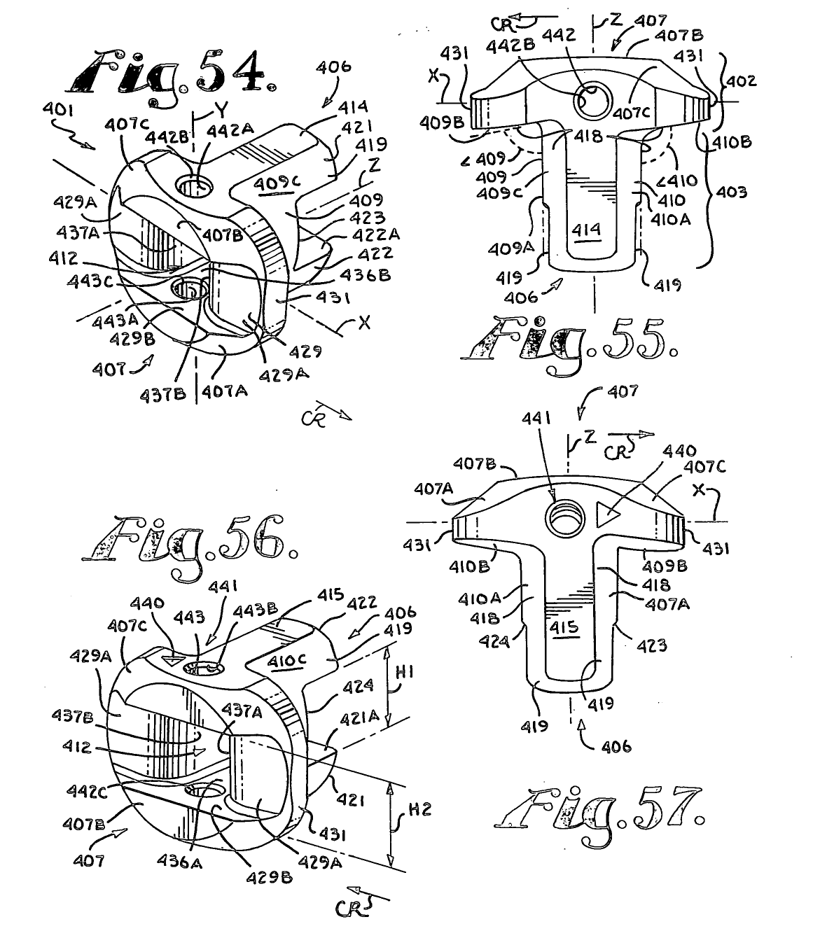

With reference to FIGS. 54-62, the illustrated interspinous process spacer 401 is shaped somewhat like a thick, vertical cross-section of a mushroom, with a cap-like portion 402 and a stem-like portion 403. This shape is most easily seen in FIGS. 55 and 57. In FIGS. 54-58 and 60-61, with respect to an implanted spacer 401, the cranial direction is indicated by the arrow CR. It is foreseen that the spacer 401 may be installed in the illustrated orientation, such as from the left anterior side of the spine 6, or the spacer 401 may be flipped over and installed from the right anterior side of the spine 6. Spacer 401 installation is described in greater detail below, with respect to FIGS. 71-81.

FIG. 54 illustrates a spacer 401 of the present invention, including three axes, a longitudinal axis Z, a superior-inferior axis X, and an anterior-posterior axis Y. The longitudinal axis Z extends from a leading end 406 to a trailing end 407 of the spacer 401. The superior-inferior axis X extends from a first side or first abutment portion 409, or connecting surface 423, to a second side or a second abutment portion 410 or connecting surface 424. The superior-inferior axis X is perpendicular to the longitudinal axis Z. The anterior-posterior axis Y extends through the cap portion 402, from a first face 414 to a second face 415. The anterior-posterior axis Y is perpendicular to both the longitudinal and superior-inferior axes, Z and X, respectively, and coaxial with the bores 442, 443, described below. The axes X, Y and Z intersect within or adjacent to a through-channel 412, described below. When the spacer 401 is implanted, the longitudinal axis Z extends between the adjacent superior and inferior spinal processes 4 and 5, such that it is oriented from one lateral side, of the patient, to the other lateral side; the superior-inferior axis X has a superior-inferior orientation; and the anterior-posterior axis Y has an anterior-posterior orientation.

The spacer 401 may also be described with regards to planes defined by the longitudinal, superior-inferior, and anterior-posterior axis Z, X and Y. The longitudinal and superior-inferior axes Z and X define a ZX plane. The superior-inferior and anterior-posterior axes X and Y define an XY plane. The anterior-posterior and longitudinal axes Y and Z defines a YZ plane.

In addition to the leading and trailing ends 406 and 407, the spacer 401 includes a pair of abutment portions, such as the first and second abutment portions 409 and 410, or first and second sides or superior and inferior sides, a through-channel 412 disposed between the opposed abutment portions 409 and 410, and a band 60 that is receivable in the through-channel 412. In some embodiments, the band 60 is a pair of bands 60 receivable in the through-channel 412.

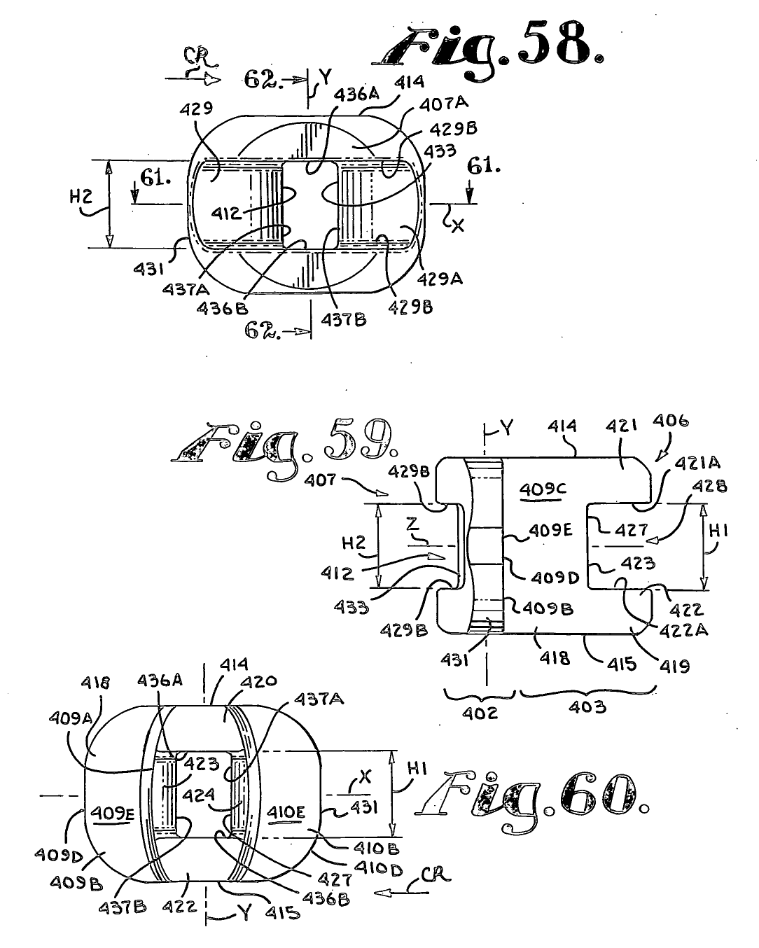

The first and second abutment portions 409 and 410 are located on opposed sides of the longitudinal axis Z, or the YZ plane. The first and second abutment portions 409 and 410 are asymmetric with respect to the YZ plane, which is most easily seen in FIGS. 55 and 57. The first and second abutment portions 409 and 410 are also symmetric, or mirror images, with respect to the ZX plane, which is most easily seen in FIGS. 58-60.

The first and second abutment portions 409 and 410 are sized and shaped for snugly and frictionally engaging adjacent superior and inferior spinal processes 4 and 5. The device 401 may be sized and shaped, or configured and arranged, such that it may be implanted from only one of the left or the right side of the spine, or such that it may be implanted from either the left or the right side of the spine. For example, the device 410 may sized and shaped so as to be implanted with the first abutment portion 409 engaging the superior spinal process 4, and the second abutment portion 410 engaging the inferior spinal process 5. In this example, since the first abutment portion 409 engages the superior spinal process 4, it may also be referred to as the superior abutment portion. Preferably, the first abutment portion 409 snugly engages the superior spinal process 4, such as by conforming to the shape of the superior spinal process 4. Similarly, the second abutment portion 410 may be referred to as the inferior abutment portion. Preferably, the inferior spinal process 5 snugly engages the inferior spinal process 5, such as by conforming to the shape of the inferior spinal process 5.

In another example, the device 410 may be implanted such that the first abutment portion 409 engages the inferior spinal process 5, and the second abutment portion 410 engages the superior spinal process 4. In this example, the first abutment portion 409 may also be referred to as the inferior abutment portion. Preferably, the first abutment portion 409 snugly engages the inferior spinal process 5, such as by conforming to the shape of the inferior spinal process 5. Similarly, the second abutment portion 410 may also be referred to as the superior abutment portion. Preferably, the superior spinal process 4 snugly engages the superior spinal process 4, such as by conforming to the shape of the superior spinal process 4.

Referring to FIGS. 55 and 57, each abutment portion includes a longitudinal portion joined with a perpendicular portion. The first abutment portion 409 includes a longitudinal portion 409A, which runs substantially parallel with the longitudinal axis Z, and a perpendicular portion 409B, which is substantially perpendicular to the longitudinal portion 409A or the longitudinal axis Z, or substantially parallel with the superior-inferior axis X. Similarly, the second abutment portion 410 includes a longitudinal portion 410A, which also runs substantially parallel with the longitudinal axis Z, and a perpendicular portion 410B, which runs substantially perpendicular to the longitudinal portion 410A or the longitudinal axis Z, or substantially parallel with the superior-inferior axis X.

Each longitudinal portion 409A and 410A includes a surface 409C and 410C, respectively, that is sized and shaped to snugly engage one of the spinous processes 4 and 5, and may include one or more of planar and curvate portions or surfaces. For example, the surfaces 409C or 410C may conform to the spinous process 4 or 5. A cross-section of the longitudinal portion 409A or 410A, which is taken simultaneously perpendicular to the longitudinal axis Z and parallel to the superior-inferior axis X is at least partially shaped like a circle, an ellipse, an oval, a super ellipse, a squoval, a squircle, or an uppercase letter D. For example, with reference to FIG. 55, the cross-section of longitudinal portion 409A is shaped like a backwards uppercase letter D, and the cross-section of longitudinal portion 410A is shaped like a forwards uppercase letter D. It is foreseen that the cross-section may include one or more generally planar portions.

Each perpendicular portion 409B and 410B extends outward, in a generally or substantially superior-inferior direction or manner, from the associated longitudinal portion 409A and 410A, respectively. When viewed from either of the first or second faces 414 or 415, respectively, the spacer 401 appears to be T-shaped or mushroom-shaped, with a cap portion 402 and a stem portion 402. The cap portion 402 includes the perpendicular portions 409B and 410B. The stem portion 403 includes the longitudinal portions 409A and 410A. Each face 414 and 415 is planar and includes a T-shaped perimeter. Each of the perpendicular portions 409B and 410B includes a perimeter portion 409D and 410D, respectively. The perimeter portion 409D and 410D may each include straight or curved portions. Further, each of the perimeter portion 409D and 410D is at least partially shaped like a circle, an ellipse, an oval, a super ellipse, a squoval, a squircle, or an upper case letter D.

As shown in FIGS. 55 and 57, the perpendicular portions 409B and 410B are generally non-symmetrical across the longitudinal axis Z or the YZ plane. Namely, the perpendicular portion 409B, or a surface 409E thereof, is slanted or slopes slightly towards the trailing end 407 and away from the leading end 406. In contrast, the perpendicular portion 410B, or a surface 410E thereof, is slanted or slopes slightly away from the trailing end 407 and towards the leading end 406. Accordingly, an angle ∠409, shown in phantom, defined by the longitudinal and perpendicular portions 409A and 409B of the first of the abutment portion 409 is an acute angle. An angle ∠410, shown in phantom, defined by the longitudinal and perpendicular portions 410A and 410B of a second of the abutment portion 410 is an obtuse angle. The angles ∠409 and ∠410 are measured relative to the longitudinal axis Z and within the ZX plane.

Each of the surfaces 409E and 410E, of each of the perpendicular portions 409B and 410B, respectively, is sized and shaped so as to snugly engage, fit against or mate with the lateral side of the adjacent spinous processes 4 and 5, when the spacer 401 is inserted between the two spinous processes 4 and 5. In order to fit closely with the lateral side of the spinous process 4 or 5, the surface 409E may be shaped to include one or more concave, convex and planar portions. For example, an inner portion of the surface 409E may be shaped, sculpted or contoured so as to be concave adjacent, or closest, to the longitudinal portion 409A, and to become progressively planar as the perpendicular portion 409B extends away from the longitudinal portion 409A.

In order to fit closely with the lateral side of the spinous process 4 or 5, the surface 410E may be shaped, sculpted or contoured so as to include one or more concave, convex and planar portions. For example, an inner portion of the surface 410E may be convex adjacent, or closest, to the longitudinal portion 410A, and become progressively planar as the perpendicular portion 410B extends away from the longitudinal portion 410A. Further, longitudinal portions 409A, 4510A smoothly curve into the associated perpendicular portions 409B and 410B, so as to form a smooth and curvate join.

The spacer 401 further includes contact and non-contact portions 418 and 419, which may be more easily discerned in FIG. 61. FIG. 61 is a schematic, or cartoon, of a cross-section of the spacer 401, when engaging the superior and inferior spinous processes 4 and 5, wherein the cross-section is taken along the ZX plane, or line 61-61 of FIG. 58. It is noted that FIG. 61 is not drawn to scale, and is for discussion purposes only.

As is described in greater detail below, when the spacer 401 is implanted, it is inserted between the spinous processes 4 and 5 from a first lateral side of the spine 6, so as to extend therebetween and then outwardly, or laterally, from the second lateral side of the spine 6. The contact portion 418 is sized and shaped for engaging the spinous processes 4 and 5. For example, the superior spinous process is engages at its lateral and caudal sides, and the inferior spinous processes is engaged at its lateral and cranial sides. The non-contact portion 419 extend laterally away from the contact portion 418, so as to not contact the spinous processes 4 and 5. Since the non-contact portion 419 extends beyond the spinous processes 4 and 5, the leading end 406, or a surface thereof, is spaced a distance from, or laterally away from, the spinous processes 4 and 5.

In FIG. 61, the spacer 401 is shown as being implanted from the right lateral side of the spine 6. Accordingly, a first portion of the contact portion 418, which is associated with the superior side 409 of the spacer 401, smoothly wraps around the superior spinous process 4, so as to engage both the lateral and caudal sides 4A and 4B. Similarly, a second portion 410 of the contact portion 418, which is associated with the inferior side of the spacer 401, smoothly wraps around the inferior spinous process 5, so as to engage both its lateral and cranial sides 5A and 5B. The non-contact portion 419 extends beyond the caudal side 4B and the cranial side 5B of the spinous process 4 and 5, respectively. The non-contact portion 419 includes a length sufficient for extending beyond the engaged pair of spinous processes 4 and 5, such that a leading surface 406A of the leading end 406 is spaced therefrom.

FIGS. 61, 80-82 illustrate the cooperative engagement of the spinous processes 4 and 5 by the abutment portions 409 and 410, or the contact portion 418. The spacer 401, shown in cross-section, is implanted from the right lateral side of the spine 6, between spinous processes 4 and 5. Thus, the stem-like portion 403 extends between the spinous processes 4 and 5 such that the abutment portion longitudinal surfaces 409C and 410C abut the inferior and superior surfaces of the spinous processes 4 and 5, respectively, and the abutment portion perpendicular surfaces 409E and 410E abut the right lateral sides of the spinous processes 4 and 5. Alternatively, if the spacer 401 is implanted from the left lateral side of the spine 6, the abutment portion longitudinal surfaces 409C and 410C will still abut the inferior and superior surfaces of the spinous processes 4 and 5, respectively, however, the abutment portion perpendicular surfaces 409E and 410E abut the left lateral sides of the spinous processes 4 and 5.

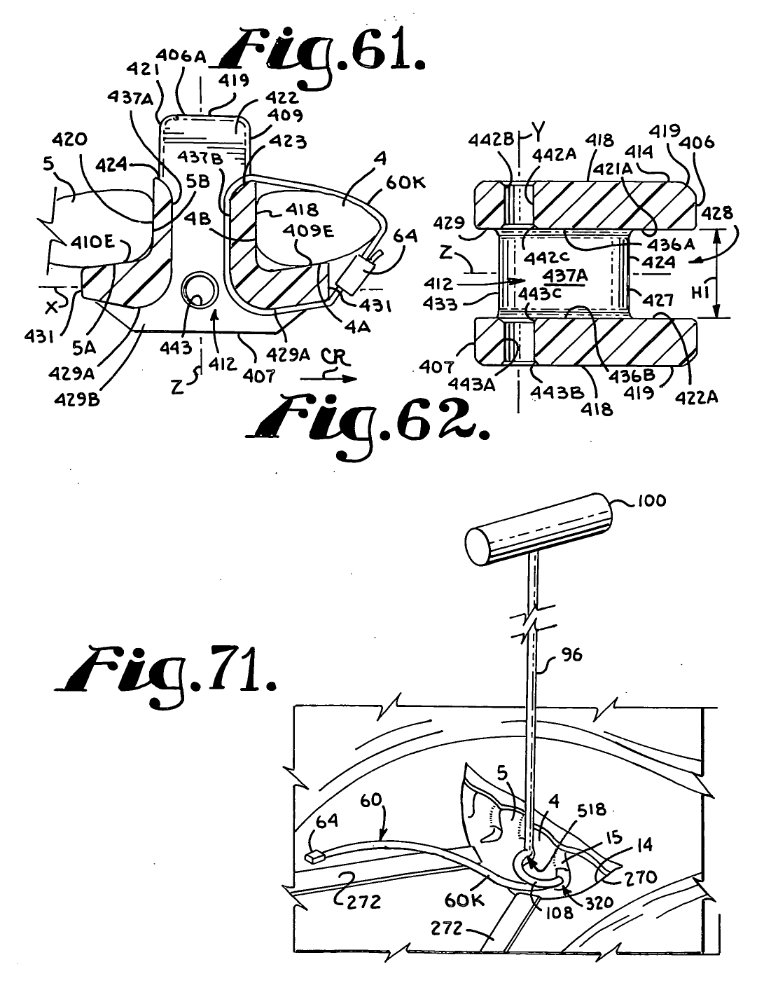

As shown in FIGS. 54 and 56, the contact portion 418 includes a curvate surface, such as surface 409C or 410C, that runs substantially, or generally, parallel with the longitudinal axis Z. The curvate surface is at least partially convex, or outwardly bulging, so as to conform to the caudal and cranial surfaces of the spinous processes 4 and 5. As shown in FIG. 60, the contact portion 418 also includes a planar or partially planar surface, such as the surfaces 409E and 410E, which extend outwardly, such as laterally extending from the curvate surfaces discussed above. Stated another way, the contact portion 418 includes a first portion that runs substantially, or nearly, parallel with the longitudinal axis Z and a second portion that runs substantially, or nearly, parallel with the superior-inferior axis X.

Referring to FIGS. 54 and 56, 59 and 62, the non-contact portion the non-contact portion 419 includes a pair of spaced legs 421 and 422. As shown in FIG. 59, the legs 421 and 422 are spaced a distance H1 that is about equal to a width of the band 60. Preferably, the distance H1 is at least slightly greater than the width of the band 60. For example, the distance H1 may be about 1, 2, 3, 4 or 5-mm greater than the width of the band 60. While it is foreseen that the distance H1 may be more than 5-mm greater than the width of the band 60, a more snug fit is preferred, so as to prevent slipping of the band 60 after device 410 implantation.

Each leg 421, 422 includes an inner leg surface 421A and 422A, respectively, wherein the inner leg surfaces 421A and 422A are opposed and spaced by the distance H1. Each inner leg surface 421A, 422A runs substantially parallel with the longitudinal axis Z, or the ZX plane. The inner leg surfaces 421A and 422A are joined by connecting surfaces 423 and 424. For example, the connecting surface 423 joins the superior sides of surfaces 421A and 422A. Similarly, the connecting surface 424 joins the inferior sides of surfaces 421A and 422A. The connecting surfaces 423 and 424 run substantially parallel with anterior-posterior axis Y, or the ZX plane, and include the height H1, described above. Further, the connecting surfaces 423 and 424 are curved so as to join the through-channel 412 with the surfaces 409C and 410C, respectively, of the first and second abutment portions 409 and 410, respectively. For example, the connecting surface 423 joins the through-channel side surface 437B with the surface 409C. Similarly, the connecting surface 424 joins the through-channel side surface 437A with the surface 410C.

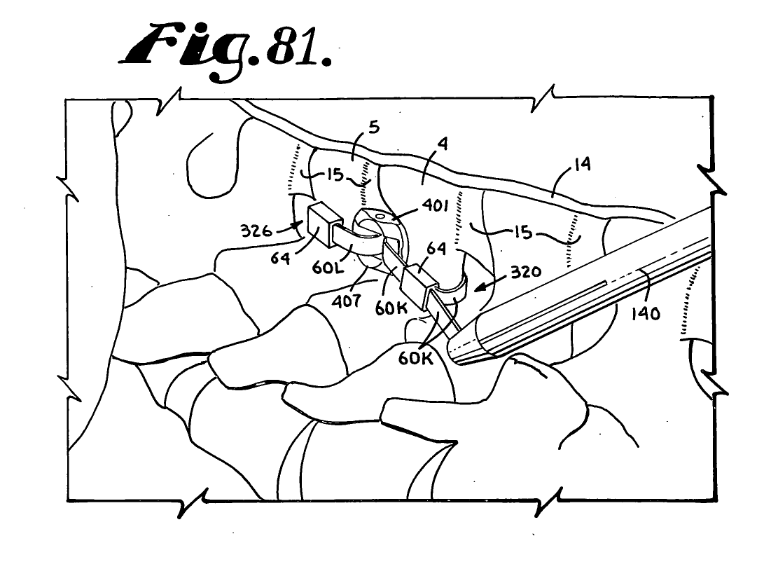

As shown in FIG. 80, the band 60K engages the connecting surface 423 and the band 60L engages the connecting surface 424. The connecting surfaces 423 and 424 are smooth and curved, so as to not substantially abrade or fray the bands 60K, 60L. It is foreseen that in some circumstances the joining surfaces 423 and 424 may be beveled or a plane that runs parallel with the XY plane. In such circumstances, the edges of the joining surfaces 423 and 424 may be sharp corners, curvate or rounded off. It is foreseen that the connecting surfaces 423, 424 may be roughened or textured so as to increase friction between the surfaces 423, 424 and the bands 60K, 60L.

A first or leading through-channel opening 427 is defined by at least a portion of each of the surfaces 423, 424, 421A and 422A. The opening 427 is adapted to receive the band 60 therethrough. Thus, as shown in FIG. 61, the band 60K extends from the through-channel 412, out of the opening 427, and wraps around the connecting surface 423.

As shown in FIGS. 59, 60 and 62, the inner surfaces 421A and 422A and the connecting surfaces 423 and 434 also define a recessed portion, generally 428. The recessed portion 428 is associated with the legs 421 and 422. The recessed portion 428 is also associated with the non-contact portion 419.

Referring now to FIGS. 54-58, the trailing end 407 is located opposed to the leading end 406. The trailing end 407 includes a trailing surface 407A with planar and curvate portions 407B and 407C. The planar portion 407B runs substantially parallel with a XY plane, and is bisected by a band guide portion 429, described below. A discontinuously circular or a discontinuously ovular perimeter portion joins the planar portion 407B with the curvate portion 407C. The curvate portion 407C slopes outward and forward, or outward and towards the leading end 406, and joins with the trailing ends of the first and second faces 414 and 415.

A pair of connecting portions 431 join the lateral ends or sides of the curvate portion 407C with the perpendicular portions 409B and 410C, of the abutment portions 409 and 410, respectively. Accordingly, the trailing surface 407A is spaced from the abutment portions 409 and 410, such as by the width of the connecting portions 431. One of the connecting portions 431 joins the superior cap portion ends of the faces 414 and 415. The other connecting portion 431 joins the inferior cap portion ends of the faces 414 and 415. The connecting portions 431 each include a cross-section, taken parallel with the XY plane, that is at least a partially shaped like a circle, an ellipse, an oval, a super ellipse, a squoval, a squircle, or an upper case letter D.

Referring to FIGS. 54 and 56, 58, 59 and 62, the trailing end 407 includes a band guide portion 429 that is sized, shaped and located so as to receive and engages a band 60 therein. The band guide portion 429 runs substantially parallel with the superior-inferior axis X, and bisects the planar portion 407B, and optionally the curvate portion 407C, of the trailing end 407. The band guide portion 429 includes a recessed surface 429A that frictionally engages the received band 60. The band guide portion 429 includes a pair of coplanar band-engagement surfaces 429A that run generally parallel with the XY plane. The surfaces 429A are smoothly and curvingly joined with the through-channel side surfaces 437A and 437B. A band 60K is received from the through-channel 412 and then extends outwardly along the recessed surface 429A, and frictionally engages the recessed surface 429A. The recessed surface 429A includes a height H2 that is about equal to the width of the band 60K, or equal to the height H1. Preferably, the height H2 is at least slightly greater than the width of the band 60K, such as 1, 2, 3, 4, or 5-mm wider. While it is foreseen that the height H2 may be more than 5-mm greater than the width of the band 60K, a more snug fit is preferred.

The band guide portion 429 further includes opposed inner surfaces 429B, or walls, which extend perpendicularly rearward from the recessed surface 429A, and are substantially parallel with the ZX plane. The inner surfaces 429B are located so as to join the anterior and posterior sides or edges of the recessed surface 429A. The joins between the recessed surface 429A with each of the inner surfaces 429B may be smooth and curved, beveled, or may include sharp or rounded corners. Together, the recessed and inner surfaces 429A and 429B define a contoured recess that receives the band 60 from the through-channel 412, and frictionally and/or stericly secures the band 60 laterally outward configuration or orientation.

The second or trailing through-channel opening 433 is associated with the band guide portion 429. The opening 433 is adapted to receive the band 60 from the through-channel 412, such that the band 60 may be secured in the band guide portion 429. At least a portion of the recessed and inner surfaces 429A and 429B, of the band guide portion 429, define the opening 433. Accordingly, the opening 433 is substantially rectangularly shaped and substantially parallel with the XY plane. The opening 433 provides communication between the band guide portion 429 and the through-channel 412, such that the band guide portion 429 receives the band 60 from the through-channel 412.

Referring now to FIGS. 54 and 56 and 58-62, the spacer 401 includes a through-channel 412 that is sized, shaped and located so as to receive one or more bands 60 therethrough. As elsewhere herein, the bands 60 secure the spacer 401 between the spinous processes 4, 5. The through-channel 412 runs substantially parallel with the longitudinal axis Z, from the leading or first opening 427 to the trailing or second opening 433. The through-channel 412 includes a rectangularly shaped cross-section, wherein the cross-section is taken parallel with the XY plane. The through-channel 412 may also include a second rectangularly shaped cross-section, which is taken parallel with the YZ plane.

The through-channel 412 includes two pairs of spaced or opposed inner walls, all of which run parallel with the longitudinal axis Z. The first pair of inner walls includes a first or upper inner wall 436A, and a second or lower inner wall 436B. As used herein, the terms “upper” and “lower” refer to the orientation of the spacer 401 shown in FIG. 54. The upper and lower inner walls 436A and 436B are spaced and substantially parallel with each other and run parallel with the ZX plane. The upper inner wall 436A is integral with one of the band guide portion inner surfaces 429B, and with the first leg inner surface 421A. Similarly, the lower inner wall 436B is integral with the other inner surface 429B, and with the second leg inner surface 422A. The upper and lower inner walls 436A and 436B are spaced a distance H1, or H2, that is slightly greater than a width of the band 60. The upper and lower inner walls 436A and 436B are substantially planar. However, it is foreseen that one or both of the upper and lower inner walls 436A and 436B may include non-planar portions.

The second pair of inner walls includes a third or left inner wall 437A, and a fourth or right inner wall 437B. As used herein, the terms “left” and “right” refer to the orientation of the spacer 401 shown in FIG. 54. The left and right inner walls 437A and 437B are substantially parallel with each other and run parallel with the YZ plane. The left and right inner walls 437A and 437B are spaced a distance equal to the width of the upper and lower inner walls 436A and 436B. The left and right inner walls 437A and 437B are substantially planar. It is foreseen that one or both of the left and right inner walls 437A and 437B may include non-planar portions.

Each of the inner walls 436A, 436B, 437A and 437B is joined with two adjacent walls, so as to provide the rectangular cross-section that is parallel with the XY plane. For example, upper inner wall 436A is joined with left and right inner walls 437A and 437B, wherein the left and right inner walls 437A and 437B are located on opposed sides of the YZ plane. Similarly, lower inner wall 436B is joined with left and right inner walls 437A and 437B, wherein the left and right inner walls 437A and 437B are located on opposed sides of the YZ plane. In another example, the left inner wall 437A is joined with the upper and lower inner walls 436A and 436B, such that the upper and lower inner walls 436A and 436B are located on opposed sides of the ZX plane. Similarly, the right inner wall 437B is joined with the upper and lower inner walls 436A and 436B, such that the upper and lower inner walls 436A and 436B are located on opposed sides of the ZX plane. The intersections, or joins, of the walls 436A, 436B, 437A and 437B, may be curves, beveled or sharply angular.

The left inner wall 437A joins the connecting surface 424, at the first through-channel opening 427. The connecting surface 424 curves around, about, or outwardly, or about 180°, so as to join the surface 410C of the longitudinal portion 410A, of the second abutment portion 410. Similarly, the right inner wall 437B joins the connecting surface 423. The connecting surface 423 curves around, about, or outwardly, or about 180°, so as to join the surface 409C of the longitudinal portion 409A, of the first abutment portion 409.

Each of the left and right inner walls 437A and 437B joins one of the band guide portion recessed portions 429A, at the second through-channel opening 433. Each join or intersection of each of the left and right inner walls 437A and 437B with one of the recessed portions 429A is smoothly curved about 90°, such that each of the left and right inner walls 437A and 437B is substantially perpendicular with the joined recessed portion 429A.

Referring to FIGS. 56 and 57, the spacer 401 includes one or more indicia 440 that are associated with the orientation of the spacer 401 during and after installation. Namely, the indicia 440 indicate to the surgeon the correct way to hold the spacer 401, or the orientation in which to hold the spacer 401, while it is being implanted between the spinous processes 4, 5. Numerous types of indicia 440 may be used with the spacer 401. Suitable indicia 440 include but are not limited to printed shapes or symbols, color-coded shapes or symbols, engraved shapes or symbols, and embossed shapes or symbols. For example, the indicia 440 may be one or more letters, words, numbers, shaped or symbols. In the illustrated invention, the indica 440 is a triangle located on the face 415 so as to be located adjacent to the opening 443B. One corner of the triangle 440 is pointed towards the connecting portion 431 associated with the superior abutment portion 409, in a manner similar to that of an arrow head. In some circumstances, the triangle may point to the cranium during implantation, which direction is indicated by the arrow CR. Alternatively, an entire surface or portion of the device may be colored or textured, so as to indicate a preferred device orientation.

It is foreseen that indicia 440 can be provided by a variety of means known in the art, such as but not limited to engraved indicia, molded indicia, painting, notches, break-off tabs, snaps, buttons, and the like. Further, any indicia 440 with suitable meaning to the surgeon can be used, including but not limited to arrows, dots, bumps, pictograms, words, and the like. In an exemplary embodiment, an arrow, which is oriented so as to point towards the patient's head when the spacer 401 is in the correct orientation for implantation, is engraved on one or both side surfaces 414 and 415. In another exemplary embodiment, the triangle 482 depicted in FIGS. 56 and 57 is replaced by a pictogram of a human head. In still another exemplary embodiment, the superior connecting portion 431 is colored or painted, so as to indicated that the painted connecting portion 431 should be located closer to the cranium, than the non-colored connecting portion 431, during implantation.

In the FIGS. 54-57, the indicia 440 is an engraved, embossed or colored triangle that indicates the cranial direction CR. During implantation, the surgeon holds the spacer 401 such that the indicia 440 indicates the superior side of the spine 6. For example, the indicia 440 may point towards the cranium. It is foreseen that the indicia 440 may be located so as to indicate the inferior side of the spine 6. Alternatively, the superior abutment portion, such as but not limited to abutment portion 409, such that the indicia 440 is located next to the superior vertebra 4, after implantation. In still another example, the inferior abutment portion, such as but not limited to abutment portion 410, such that the indicia 440 is located next to the inferior vertebra 5, after implantation. Numerous locations of the indicia 440 are foreseen.

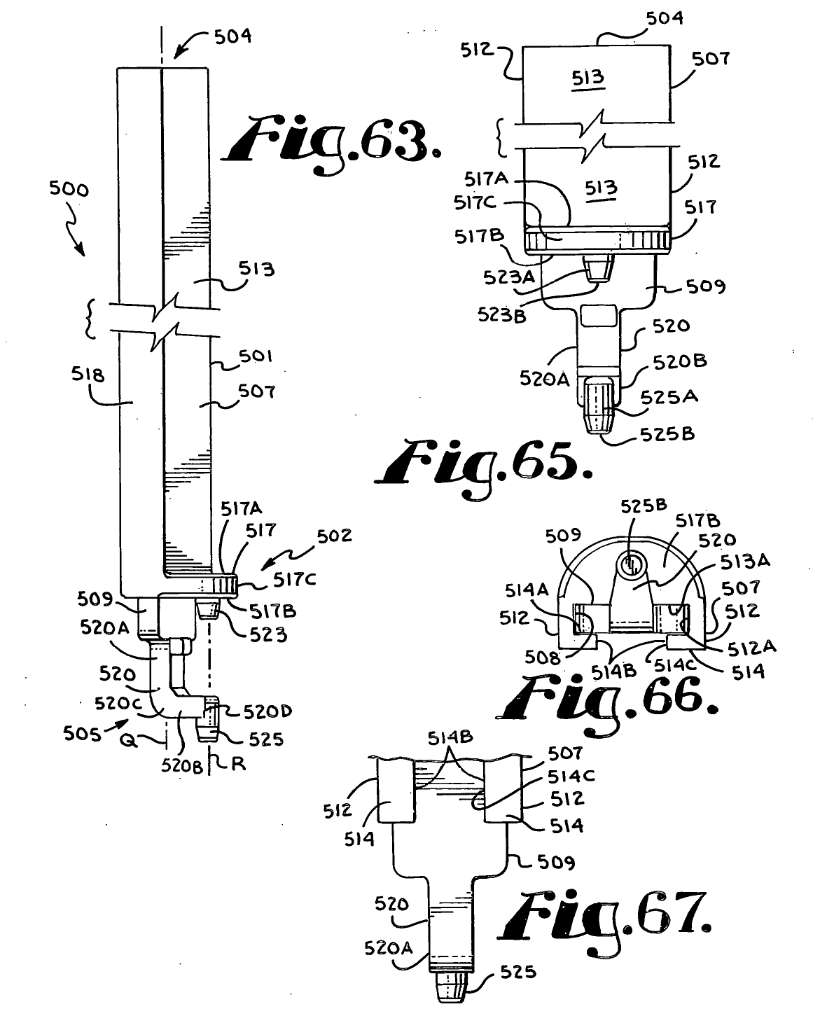

Referring to FIGS. 54-57, 61-62, 69, and 70, the spacer 401 includes a tool-engagement structure, generally 441, which is sized and shaped to be engaged by an installation tool, generally 500. The installation tool 500 is described below with regards to FIGS. 63-70 and 79. The tool-engagement structure 441 includes one or more tool-engagement bores 442 and 443 associated with or located in one or both of the faces 414 and 415. Preferably, the spacer 401 includes a pair of tool-engagement bore 442, 443 wherein one of the bores 442, 443 is associated with or located in each of the faces 414 and 415. For example, a first tool-engagement bore 442 is associated with or located in the first face 414. A second tool-engagement bore 443 is associated with or located in the second face 415. The tool-engagement bores 442, 443 are located so as to be coaxial with the anterior-superior axis Y, such as is shown in FIG. 62. The tool-engagement bores 442, 443 are also spaced along a length of the anterior-superior axis Y. The tool-engagement bores 442, 443 are further spaced along the anterior-superior axis Y, so as to be located on opposite sides of the ZX plane.

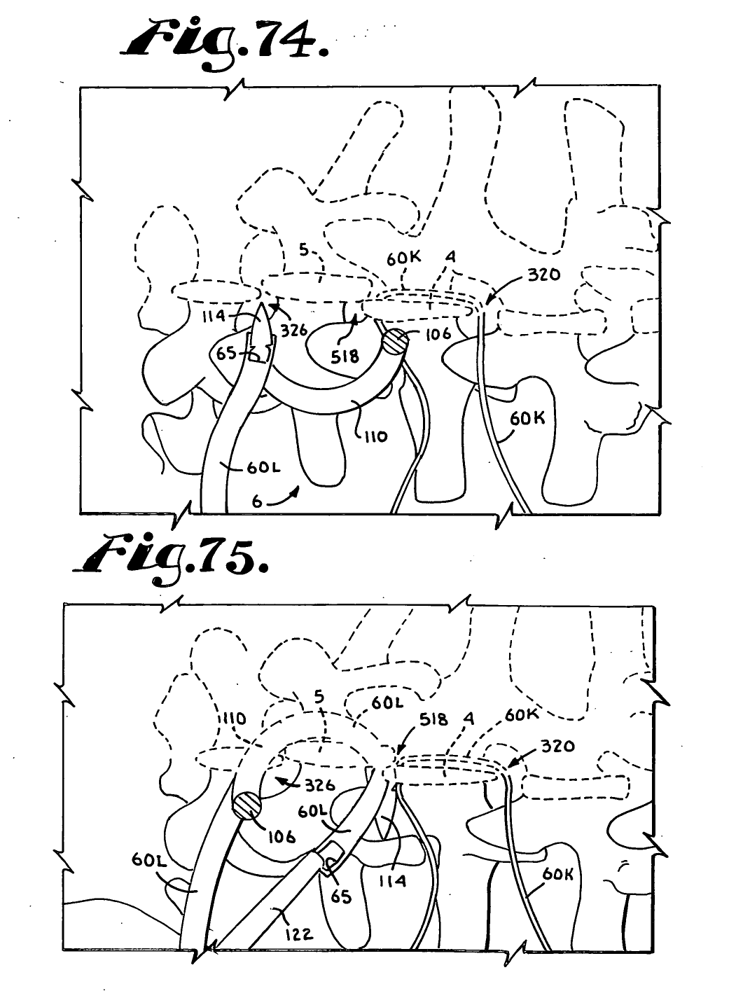

Referring to FIG. 62, each tool-engagement bore 442 and 443 includes a smooth, cylindrically shaped inner surface 442A and 443A, respectively. The inner surface 442A of the first tool-engagement bore 442 extends from an exterior opening 442B located in the first face 414 to an interior opening 442C located in the upper inner wall 436A of the through-channel 412. The inner surface 443A of the second tool-engagement bore 443 extends from an exterior opening 443B located in the second face 415 to an interior opening 443C located in the lower inner wall 436B of the through-channel 412. Each of the tool-engagement bores 442 and 443 is sized, shaped and located so as to be engaged by the installation tool 500, such as described below with regards to FIGS. 69-70.

In an exemplary embodiment, the spacer 401 for placement between a pair of spinous processes 4, 5 includes a cap portion 402 and a stem portion 403. The cap portion 402 extends outwardly from the longitudinal axis Z and along the superior-inferior axis X. The cap portion 402 includes the trailing end 407, as well as the first and second engagement portions 409B and 410B. The trailing end 407 includes the trailing surface 407B and the band engagement surface 429A. Each of the surfaces 407B and 429A run substantially parallel with the superior-inferior axis X, or with the XY plane. The first and second engagement portions 409B and 410B each include the engagement surface 409E and 410E, respectively. The engagement surface 409E and 410E are sized, shaped and located so as to engage the lateral sides of the spinous process 4, 5.

The stem portion 403 extends perpendicularly from the cap portion 402, along the longitudinal axis Z. The stem portion 403 includes the leading surface 406A, the third and fourth engagement portions 409A and 410A, the band-receiving channel 412, and the band 60. The first and third engagement portions 409B and 409A, respectively, are joined together or integral, so as to provide a smooth and curvate transition therebetween. Similarly, the second and fourth engagement portions 410B and 410A, respectively, are joined together or integral, so as to provide a smooth and curvate transition therebetween.

The band-receiving channel 412 extends along the longitudinal axis Z, from about the cap trailing end 407 to about the stem leading surface 406. The band-receiving channel 412 is sized and shaped to receive the band 60 therethrough. For example, the band-receiving channel 412 includes a height of about H1 or H2, which is about equal to the width of the band 60, and such that the band 60 may be smoothly or easily received therein. The band 60 sized and shaped to extend through the band-receiving channel 412 and around a first spinous process 4 or 5. For example, the band 60, or 60K, cooperatively abuts or engages the spinous process 4 or 5 with the surface of one of the engagement portions 409B or 410B.

In a further exemplary embodiment, the spacer 401 includes a second band 60, or 60L, sized and shaped to extend through the band-receiving channel 412 and around a second spinous process 4 or 5 in cooperative abutment with a surface of the second engagement portion 410B. The stem 403 is sized and shaped for distraction of the first and second spinous processes 4 and 5. The spacer 401 further includes a tool-engagement structure 441. The spacer 401 may also include the indicia 440 associated with an orientation of device installation, such as described above.

FIGS. 63-70 illustrate an installation tool, generally 500, that is sized, shaped and arranged for engaging, or holding, the interspinous process spacer 401, such that the spacer 401 may be installed, implanted or inserted between the adjacent spinous processes 4, 5, such as is described below with regards to FIGS. 69-70 and 79. The installation tool 500 is sized, shaped and arranged for releasably engaging or mating with a spacer 401. While only spacer 401 is depicted as including the tool-engagement structure 441, any other spacer described herein, such as spacers 1 and 201, can be manufactured with a suitable tool-engagement structure 441. Further, many other interspinous process spacers known in the art can be adapted to include suitable tool-engagement structures 441, such that these spacers can be implanted using the installation tool 500 of the illustrated invention.

The installation tool 500 of the present invention includes an elongate handle portion 501 and an engagement subassembly, generally 502. The handle portion 501 extends along a longitudinal axis Q, from a top end, generally 504, to a bottom end, generally 505. The handle portion 501 includes an outer or stationary handle portion 507 with a through-channel 508 located therein, and an inner or movable handle portion 509 slidingly received in the through-channel 508. The engagement subassembly 502 is located at the bottom end 505 of the handle portion 501. Thus, a portion of the engagement subassembly 502 is located at the bottom ends of each of the inner and outer handle portions 509 and 507, respectively. At least a portion of the engagement subassembly 502 extends perpendicularly outward, or forward from the handle bottom end 505, with respect to the longitudinal axis Q. The engagement subassembly 502 is sized, shaped and arranged for engaging and implanting the spacer 401 between the spinous processes 4, 5.

Referring to FIGS. 63, the outer handle portion 507 is shaped substantially like an elongate tube with a rectangular cross-section, which is open at the top and bottom ends 504 and 505. The outer handle portion 507 includes a pair of side walls 512 joined by a front wall 513. The side walls 512 are substantially planar. The side walls 512 are parallel with each other and spaced a distance equal to a width of the front wall 513, or a distance sufficient for the inner handle portion 509 to be receive within the through-channel 508.

To the rear, the outer handle portion 507 includes a pair of spaced and parallel back walls 514. The back walls 514 run parallel with the front wall 513. Each of the back walls 514 is joined with an adjacent side wall 512 at an angle of about 90°. Thus, the walls 512, 513 and 514 define a substantially rectangular cross-section of the outer handle portion 507, wherein the cross-section is taken perpendicular to the longitudinal axis Q. The walls 512, 513 and 514 further define the through-channel 508, which slidingly receives the inner handle 509.

Each of the wall 512, 513 and 514 includes a smooth and planar inner surface. The front wall 513 includes a front inner surface 513A. Each side wall 512 includes a side inner surface 512A. Similarly, each of the back walls 514 includes a back inner surface 514A. The front inner surface 513A is joined with the side inner surfaces 512A, wherein the inner surfaces associated with each join define an angle of about 90°. Similarly, each of the side inner surfaces 512A is joined with an back inner surface 514A, such that the back inner surfaces 514A are perpendicular to the side inner surfaces 512A and parallel with the front inner surface 513A. Accordingly, the inner surfaces 512A, 513A and 514A, define the through-channel 508, which includes a substantially rectangular cross-section, taken perpendicular to the longitudinal axis Q. The inner surfaces 512A, 513A and 514A are substantially smooth and planar, such that the inner handle 509 may be slidingly received into or by, or slidingly engaged by, the through-channel 508.

Referring to FIGS. 66-68, the back walls 514 include parallel and spaced facing surfaces 514B. The facing surfaces 514B extend from the top end 504 to the bottom end 505 of the outer handle portion 507, and are spaced so as to define an elongate, rectangular opening 514C extending from the top 504 to the bottom 505 of the outer handle portion 507. The rectangular opening 514C is in communication with the through-channel 508, thereby providing access to the inner handle portion 509, when it is received within the through-channel 508. For example, the surgeon may insert a finger through the opening 514C and so as to press against the inner handle portion 509, so as to manipulate the inner handle portion 509 upward or downward, or so as to maintain the inner handle portion 509 in a substantially steady or constant position. Alternatively, the opening 514C may be sufficiently narrow so as to prevent significant contact of the inner handle portion 509 by the surgeon. Numerous opening 514C widths are foreseen.

The inner handle portion 509 is sized and shaped so as to be slidingly received in the through-channel 508. The inner handle portion 509 includes sides that are substantially planar and smooth. It is foreseen that the surfaces of the inner handle portion 509, and/or of the through-channel 508, may be roughened, knurled, textured, or even ratcheted, so as to reduce sliding therebetween. Alternatively, the surfaces of the inner handle portion 509, and/or of the through-channel 508, may be polished or covered with a hard, slick coating so as to reduce friction therebetween.

The engagement subassembly 502 includes a foot 517 associated with the outer handle portion 507 and an arm 520 associated with the inner handle portion 508. The foot 517 may be integral or non-integral with the outer handle portion 507. Similarly, the arm 520 may be integral or non-integral with the inner handle portion 508.

The foot 517 extends perpendicularly and forwardly outward from the bottom end 505 of the outer handle portion 507. The foot 517 includes substantially planar and parallel upper and lower surfaces 517A and 517B, respectively, joined by a substantially curvate intermediate surface 517C. A cross-section of the foot 517, taken perpendicular to the axis Q, is substantially circular or ovular, or shaped like an upper case letter D. It is foreseen that the foot 517 may include at least partially rectangular or triangular cross-sections, or the cross-section may include both planar and curvate portions. The foot 517 is shaped, sized and located so as to releaseably engage the spacer 401. For example, the foot 517 lower surface 517B may releasably and frictionally engage the spacer upper face 414.

The foot 517 includes a first or upper pin, peg or finger 523 that extends downwardly from the lower surface 517B. The foot upper pin 523 is sized, shaped and located to as to releasably engage the tool engagement structure 441 of the spacer 401. For example, the upper pin 523 is sized, shaped and located so as to releasably mate with the first or upper bore 442 of the tool engagement structure 441. The upper pin 523 includes a longitudinal axis R that runs substantially parallel with the longitudinal axis Q of the handle portion 501. When the installation tool 500 engages the spacer 401, the upper pin longitudinal axis R is coaxial with the spacer anterior-posterior axis Y. The upper pin 523 includes an outer surface 523A and an end surface 523B. The outer surface 523A includes at least one of a cylindrical portion and a conical portion. The conical portion may be truncated, such that the end surface 523B is substantially blunt. For example, in FIG. 63, the end surface 523A is substantially planar, perpendicular to the axis R, and includes a substantially circular perimeter. Alternatively, the end surface 523B may be rounded, convex, dome-shaped, or pointed.

The arm 520 extends downwardly from the inner handle 509, and includes a vertical portion 520A joined with a horizontal portion 520B. The vertical and horizontal portions 520A and 520B are joined at an elbow portion 520C. The vertical and horizontal portions 520A and 520B may each include a substantially rectangular cross-sections. It is foreseen that on or both of the and horizontal portions 520A and 520B may include a substantially circular or ovular cross-sections. The arm 520 may be integral with the inner handle 509, or the arm 520 may be fabricated separately and then attached or secured to the bottom end of the inner handle 509. The vertical portion 520A extends longitudinally downward from the bottom end of the inner handle 509, such as substantially parallel with the handle longitudinal axis Q. The elbow portion 520C includes a bend that is adapted such that the horizontal portion 520B extends forwardly from the lower end of the vertical portion 520A. In the illustrated invention, the bend includes an angle of about 90°. It is foreseen that the bend may include angles greater or less than about 90°. The horizontal portion 520B extends forwardly from the elbow 520C and runs substantially perpendicular to the longitudinal axis Q. The vertical portion 520B includes a length sufficient to space the horizontal portion 520B a distance downward from the foot lower surface 517B.

At a forward end 520D of the horizontal portion 520B, the arm 520 includes a second or lower pin, peg or finger 525. The lower pin 525 extends downwardly from the from the end 520D, or parallel with the axis Q, and is sized, shaped and located to as to releasably engage the tool engagement structure 441 of the spacer 401. For example, the lower pin 525 is sized, shaped and located so as to releasably mate with the second or lower bore 443 of the tool engagement structure 441. The lower pin 525 includes a longitudinal axis R that runs substantially parallel with the handle portion longitudinal axis Q. As shown in FIGS. 63-64 and 69, the upper and lower pins 523 and 525 are spaced along the axis R and co-linear or coaxial. The lower pin 525 includes an outer surface 525A and an end surface 525B. The outer surface 525A includes at least one of a cylindrical portion and a conical portion. The conical portion may be truncated, such that the end surface 525B, of the lower pin 525, is substantially blunt. For example, in FIG. 63, the end surface 525A is substantially planar, perpendicular to the axis R, and includes a substantially circular perimeter. Alternatively, the end surface 525B may be rounded, convex, dome-shaped, or pointed.

The installation tool 500 includes two positions or configurations associated with releasable engagement of the spacer 401. The installation tool 500 may be manipulated by the doctor, so as to move the tool 500 between the first and second positions.

The first position, shown in FIG. 69, is associated with an initial step of engaging the spacer 401, and with releasing the spacer 401. The first position may be referred to as a “closed” position. When in the first position, the inner handle portion 509 is slid upward with respect to the outer handle portion 507. For example, when the tool 500 is in the first position, the top surfaces of the inner and outer handle portions 509 and 507 may be coplanar. In the first position, the first and second pins 523 and 525 are spaced along axis R by a first distance T1. The foot lower surface 517B may releasably engage the spacer second face 415, and the upper pin 523 may be inserted through the opening 422B and into the upper bore 422. The upper pin 523 is substantially immobile with respect to the foot 517. The upper pin 523 extends at least partially through the upper bore 422. The upper pin outer surface 523A may releasably, and optionally frictionally, engage the upper bore inner surface 422A.

As the upper pin 523 is manipulated so as to engage the upper bore 422, the lower pin 525 may be removably inserted into the spacer through-channel 412. For example, the lower pin 525 and a portion of the arm horizontal portion 520B may be inserted into the through-channel 412 by the second opening 433. Accordingly, the lower pin 525 includes a diameter that is at least slightly less than the width of the through-channel 412, or the widths of upper and lower inner walls 426A and 436B of the through-channel 412. Similarly, the arm horizontal portion 520B includes a width that is at least slightly less than the width of the through-channel 412, or the widths of upper and lower inner walls 426A and 436B of the through-channel 412. The length of the lower pin 525 is sufficiently less than the height H1 or H2 of the through-channel 412, such that the lower pin 525 easily fits between the upper and lower inner walls 426A and 436B.

The second position, shown in FIG. 70, is associated with fully engaging, holding or grasping the spacer 401. When in the second position, the tool 500 securely and releasably engages or holds the spacer 401, such that the spacer 401 may be implanted, such as is described below. The second position, when the tool 500 fully engages the spacer, may be referred to as an “open” position.

When in the second position, the upper and lower pins 523 and 525 are spaced along axis R by a second distance T2, wherein T2 is greater than T1. While the upper pin 523 is still releasably engaged with the upper bore 422, the distance T2 between the upper and lower pins 523 and 525 has been sufficiently increased over the first distance T1, that the lower pin 525 is also releasably engaged with the lower bore 423, such as through the opening 423B. Further, a lower surface 520E of the arm horizontal portion 520B releasably engages the inner surface 429B of the band guide portion 429. Since the arm 520 may be moved upward and downward, with respect to the longitudinal axis Q, the lower pin 525 is mobile with respect to the foot 517 or the upper pin 523. The lower pin 525 extends at least partially through the lower bore 423. The lower pin outer surface 525A may releasably engage the lower bore inner surface 423A.

FIGS. 71-82 illustrate implantation of one or more side-loading interspinous process spacers 401 according to the invention, wherein the spacers 401 are implantable between pairs of spinous processes and attached to adjacent spinous processes utilizing bands, ties or tapes, generally 60, as described previously herein and as shown as ties 60K and 60L in the drawing figures. For exemplary purposes only, spinous processes 4 and 5 are depicted and referred to herein. However, the spacer 401 of the instant invention can be inserted between other pairs of adjacent spinous processes, if such is needed by the patient.

Implantation of the spacer 401 is similar to implantation of spacers 1 and 201. Referring to FIG. 39, initially, an incision 270 is made with a cutting tool 271 and held open with a tool 272 near the spinous processes 4, 5. The incision 270 is advantageously located laterally to the spine, on one side only; and it is not necessary to detach or section the supraspinous ligament 14 or the interspinous ligament 15 in advance of implantation of the spacer 401. Thus providing a minimally invasive procedure.

Referring to FIG. 40, if necessary, a cutting or shaving tool 274 having a rounded working surface 276 is then used to prepare an implantation site. For example, the tool 274 is inserted into the incision and small portions of the spinous processes 4, 5 and a portion 215A of the interspinous ligament 15, adjacent to an interspinous space, generally 278, into which the spacer 401 will eventually be implanted, are removed. Since the spacer 401 is not saddle-shaped, the space 278 for implantation of spacer 401 may be made slightly smaller than that required for implantation of spacers 1 and 201, advantageously allowing for the removal of even less bone and ligament.

In some circumstances, it may be necessary to determine an adequate size of spacer 401 to fit with a particular patient's interspinous process space. Thus, one or more trial spacers may be tested, such as is described above with reference to spacers 1 and 201 and FIGS. 14, 15 and 41. The trial spacer 280 is shaped similarly to the spacer 401 and includes a tool engagement structure 441, such that the installation tool 500 may be used to insert and remove the trial spacers 280. Thus the installation tool 500 cooperates with the trial spacer 280 as previously described herein to allow for measurement of the space 278 to determine the appropriately sized spacer 401.

With particular reference to FIGS. 71-75, after the trial spacer 280 is removed, the bands 60K and 60L are implanted at the implantation site 518, in preparation for implantation of the spacer 401. The bands are implanted utilizing the band insertion tools 96 and 98 previously described with reference to FIGS. 16 and 42.

As shown in FIGS. 71 and 72, the tool 96 is utilized to thread the band 60K about the spinous process 4 and through the space or site 518. The pointed tip 112 is inserted into the eyelet 65 of the band 60K and the tip 112 with the threaded band 60K are inserted into a space generally 320 located adjacent to the spinous process 4 and opposite to the prepared space 518. The handle 100 of the tool 96 is then rotated, thereby rotating the shaft 104 and in particular the semi-circular hook portion 108 in a counterclockwise direction about the spinous process 4, threading the band 60K through the interspinous space 518.

With reference to FIG. 73, a grasping tool, such as the forceps 122, is used to seize and hold the band 60K, pulling the band 60K and turning the band 60K such that a flat surface thereof is disposed about the spinous process 4 as illustrated in FIG. 74. The handle 100 of the tool 96 is then rotated in a clockwise manner to turn the hook portion 108 back out of the interspinous space 518 and then out of the space 320.

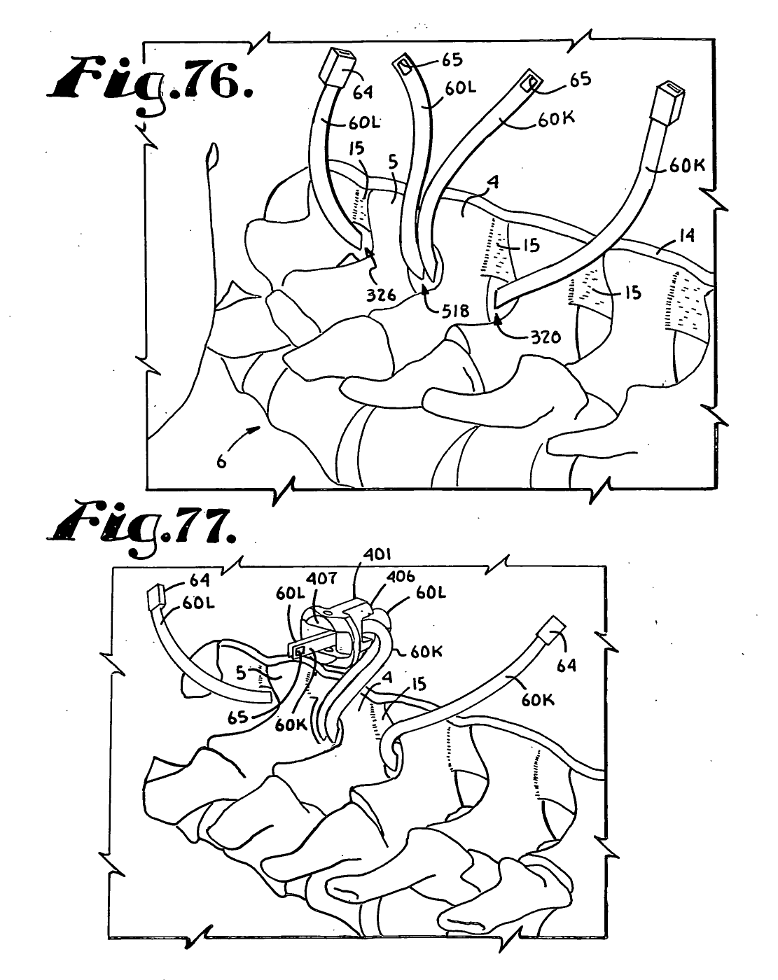

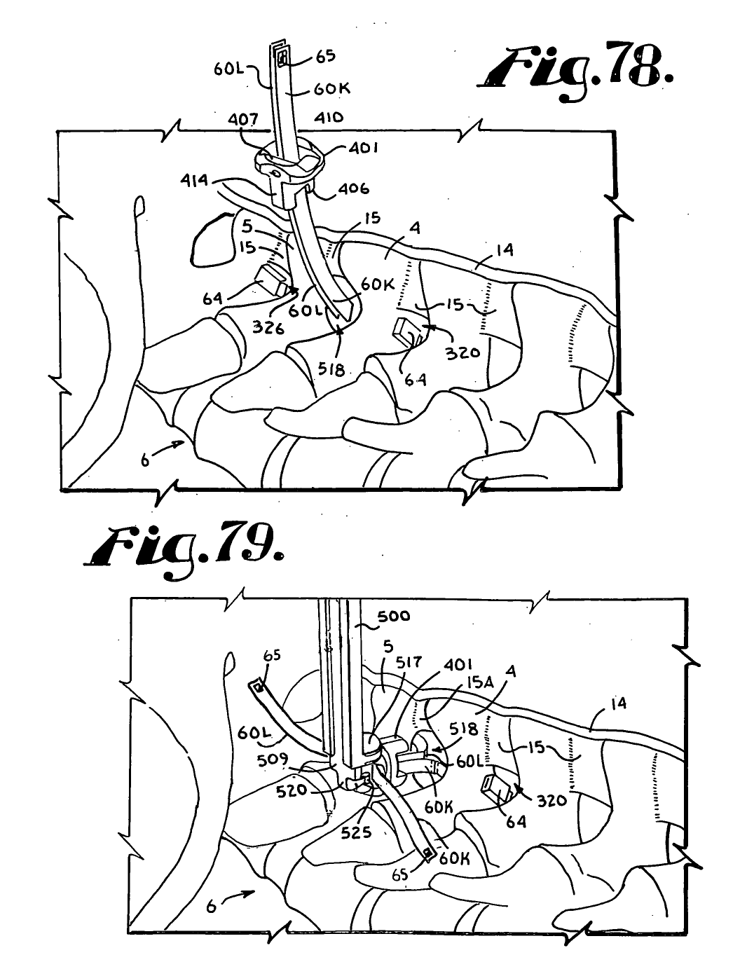

With reference to FIGS. 74-75, the band 60L is implanted in a method similar to that described above with respect to the band 60K. The pointed tip 114 of tool 98 is inserted into the eyelet 65 of the band 60L and the tip 114 with the threaded band 60L are inserted into a space generally 326, which is located adjacent to the spinous process 5, on the side opposite to the prepared space 518. The handle 102 of the tool 98 is then rotated, rotating the shaft 106 and in particular the semi-circular hook portion 110 in a clockwise direction about the spinous process 5, threading the band 60L through the interspinous space 518. With reference to FIG. 75, a grasping tool, such as the forceps 122, is used to seize and hold the band 60L, pulling the band 60L and turning the band 60L such that a flat surface thereof is disposed about the spinous process 5 as illustrated in FIGS. 76 through 79. The handle 102 of the tool 98 is then rotated in a counterclockwise manner, to turn the hook portion 110 out of the interspinous space 518 and then the space 326.

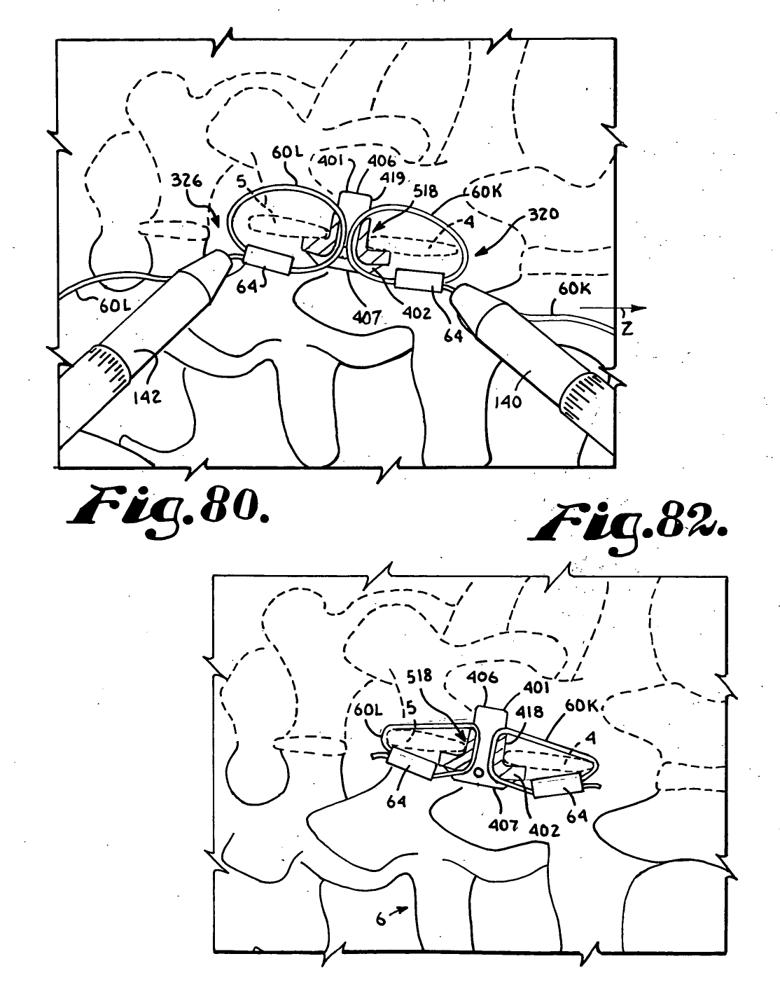

With further reference to FIGS. 76-77, the bands 60K and 60L are pulled to a desired position, shown in FIG. 76, with the eyelets 65 of the bands aligned, after which both bands 60K and 60L are threaded into the through-channel 412 of the spacer 401, such as shown in FIG. 77, at the leading end 406 and toward the trailing end 407, the flat surfaces of the bands being adjacent to the left and right inner walls 437A and 437B of the through-channel 412. With reference to FIG. 78, as the bands 60K and 60L are pulled through the through-channel 412, the fixing structures or buckles 64 are pulled into position adjacent the interspinous spaces 320 and 326.

Next, with reference to FIG. 79, the installation tool 500 is used to hold and insert the spacer 401 into the site 518 utilizing the upper and lower engagement pins 523 and 525 that releasably engage respective upper and lower bores 442 and 443 on the spacer 401. Holding the spacer 401 with the installation tool 500 is most easily seen in FIGS. 69 and 70. In FIG. 69, the upper pin 523 of the outer handle portion 507 is releasably mated with the upper bore 442 at the spacer trailing end 407. The arm horizontal portion 520B and lower pin 525 are inserted at least partially into channel 412. Then, as shown in FIG. 70, the inner handle 509 is lowered such that the lower engagement pin 525 is releasably mated with the lower bore 443 of the spacer trailing end 407.

As discussed above, and with reference to FIGS. 79-82, the stem portion 403, or the leading end 406, of the spacer 401 is inserted into the space 518 such that the contact portion 418 contacts the spinous process 4, 5, an the non-contact portion 419 extends beyond, or past, the spinous processes 4, 5. Thus, subsequent to insertion, the longitudinal surfaces 409C and 410C of the first and second abutment portions 409 and 410, respectively, or of the stem 402, cooperatively abut or contact the adjacent processes' respective inferior and superior surfaces or sides. The perpendicular surfaces 409E and 410E of the first and second abutment portions 409 and 410, respectively, or of the cap 402, simultaneously abut the adjacent processes' nearest or adjacent lateral surfaces or sides.

In some circumstances, the spacer 401 to be implanted is selected from a set of spacers 401 having a series of progressively greater or smaller sizes, or incrementally sized. For example, it may be desired to provide greater distraction between the adjacent spinous processes. Accordingly, a spacer 401 having a relatively wider, larger or thicker stem portion 403 may be selected over another spacer 401 in the series. Similarly, to provide less distraction between the spinous processes, a spacer 401 having a relatively more narrow stem portion 403 may be implanted. In another example, a set of spacers 401 can includes a series of incrementally sized spacers, such as to provide spacers 401 appropriately sized for children and adults. In yet another example, the relative size of the stem portion 403 to the cap portion 402 can be varied, to provide spacers 401 with a wide variety of shapes and sizes, such as may be required in special circumstances associated with certain stages of spinal disease progression and additionally or alternatively with certain spinal injuries.

With reference to FIGS. 80-82, once the spacer 401 is inserted into the site 518 with the contact portion 418 engaging the spinous processes 4, 5, a pair of band grasping tools 140 and 142 are utilized to thread the bands 60K and 60L through respective buckles 64 thereon and tighten the bands, the buckles 64 locking the bands snugly in place about the spinous processes 4 and 5 and band guide portion 429, or recessed portions 429A, of the spacer 401. The bands 60K and 60L are then trimmed to a desired length, such as is depicted in FIG. 82.

Removal of the spacer 401, if necessary, includes the following steps: cutting the bands 60K and 60L; removing the band portions from the interspinous spaces 320 and 326; and then removing the spacer 401 from the interspinous space 518.

It is to be understood that while certain forms of the present invention have been illustrated and described herein, it is not to be limited to the specific forms or arrangement of parts described and shown.

What is claimed and desired to be secured by Letters Patent is as follows:

1. A device for placement between a pair of spinous processes, the device comprising:
a) a longitudinal axis extending from a leading end to a trailing end;
b) a superior-inferior axis extending from a first side to a second side and being perpendicular to the longitudinal axis;
c) a pair of abutment portions, wherein the abutment portions are:
i) located on opposed sides of the longitudinal axis,
ii) asymmetric with respect to the longitudinal axis,
iii) symmetric with respect to the superior-inferior axis,
iv) associated with the first and second sides, and
v) sized and shaped for engaging adjacent superior and inferior spinal processes;
d) a through-channel disposed between the opposed abutment portions and extending parallel to the longitudinal axis; and
e) a band receivable in the through-channel.

2. The device of claim 1, wherein:
a) the longitudinal axis is adapted for extending laterally with respect to the adjacent superior and inferior spinal processes after implantation.

3. The device of claim 1, wherein:
a) the superior-inferior axis is adapted for extending superiorly to inferiorly with respect to the adjacent superior and inferior spinal processes after implantation.

4. The device of claim 1, wherein:
a) the pair of abutment portions includes a superior abutment portion and an inferior abutment portion.

5. The device of claim 4, wherein:
a) the superior abutment portion is adapted for snugly engaging the superior spinal process; and
b) the inferior abutment portion is adapted for snugly engaging the inferior spinal process.

6. The device of claim 1, wherein:
a) each of the abutment portions includes a longitudinal portion joined with a perpendicular portion.

7. The device of claim 6, wherein:
a) the longitudinal portion runs parallel with the longitudinal axis; and
b) the perpendicular portion runs parallel to the superior-inferior axis.

8. The device of claim 6, wherein:
a) each longitudinal portion has a cross-section including at least a portion of a shape selected from the group consisting of circles, ellipses, ovals, super ellipses, squovals, squircles, and D-shapes; and
b) wherein the cross-section is taken simultaneously perpendicular to the longitudinal axis and parallel to the superior-inferior axis.

9. The device of claim 6, wherein:
a) each of the perpendicular portions includes a perimeter portion including shape selected from the group consisting of partial circles, partial ellipses, partial ovals, partial super ellipses, partial squovals, partial squircles, and D-shapes.

10. The device of claim 6, wherein:
a) an angle defined by the longitudinal and perpendicular portions of a first of the abutment portion is an acute angle; and
b) an angle defined by the longitudinal and perpendicular portions of a second of the abutment portion is obtuse angle;
c) wherein the angles are measured relative to the longitudinal axis and within a plane defined by the longitudinal and superior-inferior axes.

11. The device of claim 1, further including:
a) a contact portion sized and shaped for engaging the spinous processes; and
b) a non-contact portion.

12. The device of claim 11, wherein the contact portion includes:
a) a curvate surface running parallel with the longitudinal axis; and
b) a planar surface extending outwardly therefrom.

13. The device of claim 11, wherein the non-contact portion includes:
a) a length sufficient for extending beyond an engaged pair of spinous processes.

14. The device of claim 11, wherein the contact portion includes:
a) a first portion running parallel with the longitudinal axis; and
b) a second portion running substantially parallel with the superior-inferior axis.

15. The device of claim 11, wherein the non-contact portion includes:
a) a pair of spaced legs.

16. The device of claim 15, wherein:
a) the inner legs are spaced a distance at least slightly larger than a width of the band.

17. The device of claim 15, wherein:
a) each leg includes an inner leg surface.

18. The device of claim 17, wherein:
a) the inner leg surfaces run parallel to the longitudinal axis.

19. The device of claim 17, wherein:
a) the inner leg surfaces are joined by a connecting surface.

20. The device of claim 19, wherein the connecting surface includes:
a) a width slightly greater than a width of the band.

21. The device of claim 19, wherein the connecting surface includes:
a) a first through-channel opening adapted to receive the band therethrough.

22. The device of claim 19, wherein:
a) the inner surfaces and the connecting surface define a recessed portion.

23. The device of claim 1, wherein the trailing end includes:
a) a trailing surface spaced from the abutment portions.

24. The device of claim 23, wherein:
a) the trailing surface has a planar surface and a curvate surface.

25. The device of claim 23, further including:
a) a connecting portion joining the trailing surface with the abutment portions.

26. The device of claim 25, wherein the connecting portion includes:
a) a cross-section with at least a part of a shape selected from the group consisting of circles, ellipses, ovals, super ellipses, squovals, squircles, and D-shapes; wherein
b) the cross-section is taken perpendicular to the longitudinal axis and parallel with the superior-inferior axis.

27. The device of claim 1, wherein the trailing end includes:
a) a band guide portion running parallel with the superior-inferior axis, the band guide portion being adapted for guiding a band therein.

28. The device of claim 27, wherein the band guide portion includes:
a) a recessed surface with a width slightly greater than a width of the band; wherein
b) the width of the recessed surface is measured perpendicular to the superior-inferior axis.

29. The device of claim 27, wherein the band guide portion includes:
a) second through-channel opening adapted to receive the band therein.

30. The device of claim 1, wherein the trailing end includes:
a) a planar portion running parallel with the superior-inferior axis; and
b) a curvate portion extending outwardly from the longitudinal axis.

31. The device of claim 1, wherein the trailing end includes:
a) a band guide portion sized and shaped for receiving the band;
b) wherein the band guide portion run parallel with the superior-inferior axis; and
c) wherein the band guide portion is in communication with the through-channel, so as to receive the band from the through-channel.

32. The device of claim 31, wherein the band guide portion includes a pair of co-linear band-engagement surfaces.

33. The device of claim 32, wherein:
a) the band-engagement surfaces are planar.

34. The device of claim 31, wherein the through-channel includes:
a) a second opening associated with the band guide portion.

35. The device of claim 34, wherein:
a) the second opening is substantially rectangular.

36. The device of claim 31, wherein the band guide portion includes
a) a pair of walls joined by a band-engagement surface; wherein
b) the walls are spaced a distance associated with a width of the band.

37. The device of claim 36, wherein:
a) the walls run parallel to the superior-inferior axis.

38. The device of claim 1, including
a) an indicia associated with a device installation orientation.

39. The device of claim 1, including:
a) a third side;
b) a fourth side; and
c) an anterior-posterior axis extending from the third side to the fourth side, wherein the anterior-posterior axis is perpendicular to both the longitudinal and superior-inferior axes.

40. The device of claim 39, wherein:
a) the third and fourth sides are planar.

41. The device of claim 39, wherein each of the third and fourth sides includes:
a) a generally T-shaped perimeter region.

42. The device of claim 39, wherein each of the third and fourth sides includes:
a) an opening associated with a tool-engagement bore.

43. The device of claim 39, wherein each of the third and fourth sides includes:
a) a longitudinal portion running parallel with the longitudinal axis; and
b) a perpendicular portion running parallel with superior-inferior axis.

44. The device of claim 1, further comprising:
a) a tool-engagement structure.

45. The device of claim 44, wherein:
a) the device further includes an anterior-posterior axis; and
b) the tool-engagement structure includes a pair of bores spaced along the anterior-posterior axis.

46. The device of claim 45, wherein:
a) the pair of bores are adapted for engaging an engagement structure of a tool.

47. The device of claim 44, wherein the tool-engagement structure includes:
a) a bore sized and shaped for engaging a tool.

48. The device of claim 47, wherein the bore includes a pair of bores, the pair of bores being coaxial with an anterior-posterior axis extending from a third side to a fourth side and spaced on opposed sides of the longitudinal axis.

49. The device of claim 1, wherein the device is:
a) asymmetrical with respect to the longitudinal axis; and
b) symmetrical with respect to an anterior-posterior axis.

50. The device of claim 1, wherein the band comprises:
a) a pair of bands receivable in the through-channel.

51. The device of claim 1, wherein the through-channel includes:
a) first and second inner wall running parallel with the longitudinal axis and being spaced a distance slightly greater than a width of the band;
b) third and fourth inner walls running parallel with the longitudinal axis and being joined by the first and second inner walls;
c) wherein the through-channel includes a substantially cross-section measured perpendicular to the longitudinal axis.

52. The device of claim 1, further comprising:
a) an implantation tool adapted for releasably mating with a tool-engagement portion of the device so as to implant the device between the pair of spinous processes.

53. A device for placement between a pair of spinous processes, the device comprising:
a) a cap portion having:
i) a superior-inferior axis of orientation;
ii) a trailing end with a trailing surface and a band engagement surface, each of the surfaces running parallel with the superior-inferior axis; and
iii) first and second engagement portions;
b) a stem portion extending perpendicularly from the cap portion and including:
i) a leading surface;
ii) third and fourth engagement portions, the third engagement portion being joined with the first engagement portion, and the fourth engagement portion being joined with the second engagement portion;
c) a band-receiving channel extending from the cap trailing end to the stem leading surface, and being sized and shaped to receive a band therethrough; and
d) a band sized and shaped to extend through the band-receiving channel and around a first spinous process in cooperative abutment with the first abutment portion.

54. The device of claim 53, including:
a) a second band sized and shaped to extend through the band-receiving channel and around a second spinous process in cooperative abutment with a second surface of the second abutment portion.

55. The device of claim 53, wherein:
a) the stem is sized and shaped for distraction of the first and second spinous processes.

56. The device of claim 53, further comprising:
a) a tool-engagement structure.

57. The device of claim 53, including:
a) an indicia associated with an orientation of device installation.

58. An installation tool for installing an interspinous process spacer, comprising:
a) an elongate handle extending from a top end to a bottom end along a longitudinal axis; and
b) an engagement subassembly extending perpendicularly from the bottom end of the handle with respect to the longitudinal axis, and being adapted for engaging and implant an interspinous process spacer between a pair of adjacent spinous processes of a spine.

59. The tool of claim 58, wherein the engagement subassembly includes:
a) a peg adapted for engaging a tool engagement structure of the interspinous process spacer.

60. The tool of claim 59, wherein:
a) the peg is sized and shaped for releasably mating with a bore of the tool engagement structure.

61. The tool of claim 60, wherein the peg includes:
a) a second peg sized and shaped for releasably mating with another bore of the tool engagement structure.

62. The tool of claim 59, wherein the peg includes:
a) a curvate surface and a circular cross-section; wherein
b) the cross-section is taken perpendicular with the longitudinal axis.

63. The tool of claim 59, wherein:
a) the peg extends downwardly and parallel with the handle longitudinal axis.

64. The tool of claim 63, wherein the peg includes:
a) a first peg that is immobile with respect to the handle; and
b) a mobile second peg; wherein
c) the first and second pegs are spaced and co-linear.

65. The tool of claim 58, wherein:
a) the handle includes a movable handle portion slidingly received by a stationary handle portion; and
b) the engagement subassembly includes:
i) a foot associated with the stationary handle portion and including a downwardly extending first pin; and
ii) an arm associated with the movable handle portion, the arm being spaced a distance from the foot and including a downwardly extending second pin; and wherein:
d) sliding the movable handle portion with respect to the stationary handle portion changes the distance between the foot and the arm.

66. The tool of claim 65, wherein:
a) the first and second pins are spaced along a pin central axis running parallel with the longitudinal axis of the handle.

67. The tool of claim 65, wherein:
a) the second pin is adapted for moving upward and downward with respect to the first pin.

68. The tool of claim 67, including:
a) a first position associated with the first and second pins being spaced by a first distance; and
b) a second position associated with the first and second pins being spaced by a second distance; wherein:
c) the second distance is associated with engaging a tool engagement structure of the interspinous process spacer.

69. The tool of claim 58, including:
a) an engagement position associated with an open configuration of the spacer-engagement subassembly; and
b) a non-engagement position associated with a closed configuration of the spacer-engagement subassembly.

70. An installation tool for installing a laterally-loading interspinous process spacer, comprising:
a) an outer arm having a longitudinally extending channel;
b) an inner arm slidingly engaged within the channel; and
c) a pin sized and shaped for releasably mating with a pin-receiving channel of an interspinous process spacer.

71. The tool of claim 70, wherein
a) the spacer-engaging subassembly includes a first position associated with non-engagement of said spacer; and
b) a second position associated with releasably engagement of said spacer.

72. The tool of claim 70, further comprising
a) an interspinous spaced adapted for releasable mating with the tool and lateral implantation between a pair of adjacent spinous processes.

73. A device for placement between a pair of spinous processes, the device having:
a) a longitudinal axis extending from a leading end to a trailing end;
b) a transverse superior-inferior axis extending from a first side to a second side and being substantially perpendicular to the longitudinal axis;
c) a pair of abutment portions, wherein the abutment portions:
i) are located on the leading end and the spaced opposed trailing end respectfully,
ii) are asymmetric with respect to the first side and the second side on at least one end, and
iii) are sized and shaped for engaging adjacent superior and inferior spinal processes by side loading therebetween;
d) the opposed asymmetric abutment portions cooperating with at least one band to secure the device to at least one spinous process.

74. The device of claim 73, wherein:
a) the band engages at least one side of at least one spinous process.

75. A ligament sparing device for placement between a pair of spinous processes, the device having:
a) a longitudinal axis extending from a leading end to a trailing end;
b) a transverse superior-inferior axis extending from a first side to a second side and being substantially perpendicular to the longitudinal axis;
c) a pair of abutment portions, wherein the abutment portions:
i) are located on the leading end and the spaced opposed trailing end respectfully,
ii) are asymmetric with respect to the first side and the second side on at least one end, and
iii) are sized and shaped for engaging adjacent superior and inferior spinal processes by side loading therebetween; and wherein
d) the opposed abutment portions cooperate with at least one tensionable band to secure the device to at least one spinous process.

76. A ligament sparing device for placement between a pair of spinous processes, the device having:
a) a longitudinal axis extending from a leading end to a trailing end;
b) a transverse superior-inferior axis extending from a first side to a second side and being substantially perpendicular to the longitudinal axis;
c) a pair of substantially stiff abutment portions, wherein the abutment portions:
i) are located on the leading end and the spaced opposed trailing end respectfully,
ii) are asymmetric with respect to the first side and the second side on at least one end, and
iii) are sized and shaped for engaging adjacent superior and inferior spinal processes by non-bendable side loading therebetween; and wherein
d) the opposed abutment portions cooperate with at least one tensionable band to secure the device to at least one spinous process.
